SEQUENCE LISTING (4 sequences)

SEQ ID NO: 1 - protein, 1117 aa, Homo sapiens
MARTRDRVRLLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCEVLHGSGCREAQHDSYVGLGPAAIALMILAFLLLLLVPLLLLMCHCGKGAKAFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYS

SEQ ID NO: 2 - DNA, 19 bp, Artificial Sequence
  DSG2-shRNA60
tggatgtcaatgacaatat

SEQ ID NO: 3 - DNA, 19 bp, Artificial Sequence
  DSG2-shRNA62
ccagtgttctacctaaata

SEQ ID NO: 4 - DNA, 22 bp, Artificial Sequence
  non-silencing shRNA
atctcgcttgggcgagagtaag

(19) United States
(12) Patent Application Publication
(10) Pub. No.: US 2024/0110915 A1
(43) Pub. Date: Apr. 4, 2024

(54) METHODS, PRODUCTS AND SYSTEMS FOR PROGNOSIS OF SUBJECTS SUFFERING FROM MULTIPLE MYELOMA

(71) Applicants: University of South Australia, Adelaide (AU); Central Adelaide Local Health Inc, Adelaide (AU); The University of Adelaide, Adelaide (AU)

(72) Inventors: Craig Thomas Wallington-Beddoe, Brukunga (AU); Claudine Sharon Bonder, Prospect (AU); Lisa Michelle Ebert, Bridgewater (AU)

(73) Assignees: University of South Australia, Adelaide (AU); Central Adelaide Local Health Inc, Adelaide (AU); The University of Adelaide, Adelaide (AU)

(21) Appl. No.: 18/253,153

(22) PCT Filed: Nov. 16, 2021
(86) PCT No.: PCT/AU2021/051357

§ 371 Date: May 16, 2023

(30) Foreign Application Priority Data
Nov. 16, 2020 (AU) 2020904209

(51) Int. Cl. G01N 33/574; C12Q 1/6886; G01N 15/14

(52) U.S. Cl. CPC G01N 33/57407; C12Q 1/6886; G01N 15/14; G01N 33/57492; C12Q 2600/118; C12Q 2600/158; G01N 2015/1006

(57) ABSTRACT

The present disclosure relates to methods, products and systems for the prognosis of subjects suffering from multiple myeloma. In certain embodiments, the present disclosure provides a method of prognosis for a subject suffering from multiple myeloma. The method comprises determining the level of desmoglein 2 (DSG2) in malignant plasma cells from the subject, wherein an increased level of DSG2 in the plasma cells is indicative of a poorer prognosis for the subject.

### PRIORITY CLAIM

This application claims priority to Australian Provisional Patent Application 2020904209 filed on 16 Nov. 2020, the contents of which are hereby incorporated by reference.

### FIELD

The present disclosure relates to methods, products and systems for the prognosis of subjects suffering from multiple myeloma.

### BACKGROUND

Multiple myeloma (MM) is an incurable malignancy of neoplastic antibody-secreting plasma cells (PC), with a median age at diagnosis of 69 years and a median overall survival of 6-7 years. With an age-adjusted incidence of six per 100,000 per year in the USA and Europe, it is the second most common haematological cancer. The cost to the healthcare system for each patent suffering from MM is significant.

In MM, a complex array of genetic and epigenetic changes lead to neoplastic transformation of PC, resulting in their uncontrolled growth within the bone marrow and secretion of large amounts of non-functional monoclonal antibody (known as paraprotein) into the circulation. The main clinical manifestations of MM are the development of devastating osteolytic bone lesions, bone pain, hypercalcaemia, renal insufficiency, infections and bone marrow failure. MM encompasses a spectrum of clinical variants ranging from benign monoclonal gammopathy of uncertain significance (MGUS) and smouldering/indolent MM to more aggressive, disseminated forms of MM and plasma cell leukaemia.

The past two decades have seen the introduction of novel agents that have dramatically improved overall response rates (ORR), progression-free survival (PFS) and overall survival (OS) for myeloma patients; however, disease relapse generally still occurs and the disease is currently incurable. The 10-year survival rate still remains at only 17%.

Previously, treatment of multiple myeloma relied on the use of alkylators and corticosteroids, and in some patients high dose chemotherapy with autologous stem cell transplantation. More recently, the emergence of proteasome inhibitors such as bortezomib and carfilzomib, and immunomodulatory drugs such as lenalidomide, has seen overall survival increase from 3 to 6 years. However, whilst the number of different treatment regimes has increased, MM remains an incurable disease.

The ability to stratify MM patients, based on the biology of their disease (including genetic testing) is critical in guiding appropriate therapy and clinical monitoring of an individual's risk of disease progression. For example, the t(4;14) chromosomal translocation occurs in approximately 15% of MM patients and is associated with intermediate to poor prognosis compared to patients without this translocation. t(4;14)-positive MM is characterized by rapid disease progression and disease relapse, and increased tumor dissemination, reflected by an increase in the number circulating PC in the peripheral circulation. However, genetic testing typically takes some several weeks to obtain the results and thereby delays treatment to be initiated. Currently there are no approved biomarkers available to improve assessment time.

It would be advantageous to obtain prognostic information about multiple myeloma disease and its likely progression at the time of diagnosis, for example to assist with decisions regarding personalized treatment approaches and to select appropriate treatment regimes as soon as possible.

Accordingly, there is a need for the identification of new biomarkers for multiple myeloma, and in particular to expedite the prognosis for a patient suffering from multiple myeloma, to assess their response to treatment, to assist with selecting patients for treatment, and/or to assist in the selection of appropriate treatment regimes.

### SUMMARY

The present disclosure relates to methods, products and systems for the prognosis of subjects suffering from multiple myeloma.

Certain embodiments of the present disclosure provide a method of prognosis for a subject suffering from multiple myeloma, the method comprising determining the level of desmoglein 2 (DSG2) in malignant plasma cells from the subject, wherein an increased level of DSG2 in the plasma cells is indicative of a poorer prognosis for the subject.

Certain embodiments of the present disclosure provide a method of assessing the response of a subject suffering from multiple myeloma to treatment, the method comprising determining the level of desmoglein 2 (DSG2) in malignant plasma cells from the subject, wherein an increased level of DSG2 in the plasma cells is indicative of a reduced response of the subject to the treatment.

Certain embodiments of the present disclosure provide a method of assessing clinical outcome for a subject suffering from multiple myeloma, the method comprising determining the level of desmoglein 2 (DSG2) in malignant plasma cells from the subject, wherein an increased level of DSG2 in the plasma cells is indicative of a poor clinical outcome for the subject.

Certain embodiments of the present disclosure provide a method of assessing progression of multiple myeloma in a subject, the method comprising determining the level of desmoglein 2 (DSG2) in malignant plasma cells from the subject, wherein an increased level of DSG2 in the plasma cells is indicative of progression of the multiple myeloma to a more severe stage in the subject.

Certain embodiments of the present disclosure provide a method of assessing survival of a subject suffering from multiple myeloma, the method comprising determining the level of desmoglein 2 (DSG2) in malignant plasma cells from the subject, wherein an increased level of DSG2 in the plasma cells is indicative of a reduced period of survival for the subject.

Certain embodiments of the present disclosure provide a method of selecting a treatment regime for a subject suffering from multiple myeloma, the method comprising determining the level of desmoglein 2 (DSG2) in malignant plasma cells from the subject, wherein the level of DSG2 in the plasma cells is used to select a treatment regime for the subject.

Certain embodiments of the present disclosure provide use of desmoglein 2 (DSG2) as a marker for (i) prognosis for a subject suffering from multiple myeloma, (ii) assessing response of a subject suffering from multiple myeloma to treatment, (iii) assessing clinical outcome for a subject suffering from multiple myeloma, (iv) assessing progression of multiple myeloma in a subject, (v) assessing survival of a subject suffering from multiple myeloma, and/or (vi) selecting a treatment regime for a subject suffering from multiple myeloma.

Certain embodiments of the present disclosure provide use of an antibody to desmoglein 2 (DSG2) and/or an antigen binding part thereof for (i) prognosis for a subject suffering from multiple myeloma, (ii) assessing response of a subject suffering from multiple myeloma to treatment, (iii) assessing clinical outcome for a subject suffering from multiple myeloma, (iv) assessing progression of multiple myeloma in a subject, (v) assessing survival of a subject suffering from multiple myeloma, and/or (vi) selecting a treatment regime for a subject suffering from multiple myeloma.

Certain embodiments of the present disclosure provide a kit for performing a method as described herein.

Certain embodiments of the present disclosure provide a method of prognosis for a subject suffering from multiple myeloma, the method comprising using an antibody to desmoglein 2 (DSG2) and/or an antigen binding part thereof to assess the level of DSG2 in a bone marrow sample and/or a blood sample from the subject and thereby provide a prognosis for the subject on the basis of the level of DSG2 determined.

Certain embodiments of the present disclosure provide a method of assessing the response of a subject suffering from multiple myeloma to treatment, the method comprising using an antibody to desmoglein 2 (DSG2) and/or an antigen binding part thereof to assess the level of DSG2 in a bone marrow sample and/or a blood sample from the subject and thereby assessing the response of the subject to treatment on the basis of the level of DSG2 determined.

Certain embodiments of the present disclosure provide a method of assessing clinical outcome for a subject suffering from multiple myeloma, the method comprising using an antibody to desmoglein 2 (DSG2) and/or an antigen binding part thereof to assess the level of DSG2 in a bone marrow sample and/or a blood sample from the subject and thereby assessing the clinical outcome for the subject on the basis of the level of DSG2 determined.

Certain embodiments of the present disclosure provide a method of assessing progression of multiple myeloma in a subject, the method comprising using an antibody to desmoglein 2 (DSG2) and/or an antigen binding part thereof to assess the level of DSG2 in a bone marrow sample and/or a blood sample from the subject and thereby assessing the progression of the multiple myeloma in the subject on the basis of the level of DSG2 determined.

Certain embodiments of the present disclosure provide a method of assessing survival of a subject suffering from multiple myeloma to treatment, the method comprising using an antibody to desmoglein 2 (DSG2) and/or an antigen binding part thereof to assess the level of DSG2 in a bone marrow sample and/or a blood sample from the subject and thereby assessing the survival of the subject on the basis of the level of DSG2 determined.

Certain embodiments of the present disclosure provide a method of selecting a treatment regime for a subject suffering from multiple myeloma, the method comprising using an antibody to desmoglein 2 (DSG2) and/or an antigen binding part thereof to assess the level of DSG2 in a bone marrow sample and/or a blood sample from the subject and thereby selecting the treatment regime on the basis of the level of DSG2 determined.

Certain embodiments of the present disclosure provide a computer-readable medium encoded with programming instructions executable by a computer processor means to allow the computer processor means to process data associated with the level of desmoglein 2 (DSG2) in malignant plasma cells and provide a prognosis for a subject suffering from multiple myeloma.

Certain embodiments of the present disclosure provide a computer processor means comprising a computer-readable medium as described herein.

Certain embodiments of the present disclosure provide a system for providing a prognosis for a subject suffering from multiple myeloma, the system comprising a computer processor having a computer-readable medium encoded with programming instructions executable by the computer processor means to allow the computer processor means to process data associated with the level of desmoglein 2 (DSG2) in malignant plasma cells from the subject and provide a prognosis for the subject.

Certain embodiments of the present disclosure provide a method of treating a subject suffering from multiple myeloma, the method comprising:
identifying a subject likely to be responsive to a treatment for multiple myeloma on the basis of the level of desmoglein 2 (DSG2) in malignant plasma cells; and
treating the subject so identified.

Certain embodiments of the present disclosure provide a method of selecting a specific treatment for a subject suffering from multiple myeloma, the method comprising:
determining the level of desmoglein 2 (DSG2) in malignant plasma cells from the subject; and
identifying a specific treatment for the subject on the basis of the DSG2 determined.

Certain embodiments of the present disclosure provide a method of treating a subject suffering from multiple myeloma, the method comprising:
determining the level of desmoglein 2 (DSG2) in malignant plasma cells from the subject;
identifying a specific treatment for the subject on the basis of the DSG2 determined; and
treating the subject with the specific treatment.

Certain embodiments of the present disclosure provide a method of identifying an agent for treating multiple myeloma, the method comprising:
determining the ability of a candidate agent to reduce desmoglein 2 (DSG2) dependent adhesion of malignant plasma cells to endothelial cells; and
identifying the agent as an agent for treating multiple myeloma.

Other embodiments are described herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows that DSG2 is expressed by MM-PC at the gene and protein level in a distinct subset of MM patients. (A-B): In silico analysis of publicly available microarray datasets E-MTAB-363 (A) and EGEOD-16122 (B) was performed. In these studies, RNA was extracted from CD138+ PC isolated from BM of normal donors and patients with MM or MGUS, and gene expression levels determined using the Affymetrix U133Plus2.0 platform. Threshold DSG2 expression values of 5.80 (A) and 5.62 (B) were established as described in Methods, and the proportion of DSG2+ samples above this threshold (as shown by the boxes) determined for each group. (C-E): BM or blood samples from MM patients were analysed by multi-colour flow cytometry, gating on viable CD38++/CD138+/CD45lo/CD19− PC. DSG2 expression was quantified as the difference in median fluorescence intensity (AMFI) between the DSG2-stained sample and FMO control. (C) shows all BM samples analysed. For those patients with MM-PC detectable in peripheral blood, DSG2 expression by circulating MM-PC was also assessed as shown in (D). Representative histograms are shown in (E). (F): BM trephine biopsies were stained by immunohistochemistry using control IgG, anti-CD138 or anti-DSG2 as indicated. Scale bar=100 m. FIG. 1G shows that sDSG2 is detectable in a proportion of the donors (range 0-9.5 ng·mL⁻¹). However, no discernible increase in sDSG2 was identified for the DSG2⁺ MM patients.

FIG. 2 shows DSG2 expression in a subset of human MM cell lines. (A): DSG2 gene expression values for 65 human MM cell lines were extracted from a publicly available RNA-seq dataset as described herein. Cell lines were ranked according to level of DSG2 gene expression for simplicity of visualization. (B-C): For nine of the cell lines shown in C, surface expression of DSG2 protein was assessed by flow cytometry. Examples of negative, low and high expression are shown in (B), while the relationship between gene and surface protein for all cell lines analyzed is shown in C (Spearman's correlation coefficient r=0.65).

FIG. 3 shows DSG2 expression in MM is strongly associated with reduced survival, independent of NSD2. (A): Microarray dataset GSE4581 was analysed for expression of DSG2 using probeset 1553105. Visual inspection of the data spread revealed a cluster of samples with elevated DSG2 expression. A 70/30 percentile split was applied to the data, which cleanly separated these DSG2-low and DSG2-high populations, as shown, for further analysis. (B): Overall survival was compared between the DSG2-low (lower 70%) and DSG2-high (upper 30%) subsets using Kaplan-Meier analysis. (C): Expression of DSG2 was compared between patients grouped into disease subtypes according to gene expression signatures. DSG2 expression was significantly greater in the MS subset compared to all others (Kruskall-Wallis test). (D-E): Scatterplots comparing expression of DSG2 and NSD2 genes in all samples (D) or non-MS samples only (E). Dotted lines indicate thresholds for expression based on 70^{th} percentile (DSG2) or 80th percentile (NSD2). Values represent the number of samples in each quadrant. (F): The non-MS patient cohort was stratified into DSG2-low and DSG2-high subsets and overall patient survival compared using Kaplan-Meier analysis.

FIG. 4 shows differential gene expression analysis comparing DSG2-low and DSG2-high subsets. Dataset GSE4581 was stratified into DSG2-low and DSG2-high patient subsets as per FIG. 3 and genes differentially expressed between the two groups were identified and displayed in heatmaps. Shown are analyses of the entire patient cohort (A), or only the subgroup of patients lacking MMSET expression (MS-neg; B).

FIG. 5 shows stable DSG2 knockdown does not affect the survival, migration or major signalling pathways of KMS-11 cells. (A): Western blot analysis of key signalling proteins in KMS-11 cells stably expressing nontargeting shRNA (NT) or two different DSG2-targeting shRNAs. Representative blots are shown on the left, while band densities pooled from 3 experiments are shown on the right. (B): Cell viability was determined by trypan blue counting for KMS-11 cells stably expressing NT or DSG2-targeting shRNAs, under normal culture conditions in 10% serum (left) or after overnight serum starvation (right). Data are pooled from 3 experiments. (C):KMS-11 cells expressing NT or DSG2-targeting shRNAs were serum starved for 24 h and migration/invasion capacity through Matrigel toward 20% serum was assessed after 24 h by PI staining and confocal microscopy. Representative images are shown on the left. The number of invaded cells at 60 m was quantified and pooled from 3 experiments (right).

FIG. 6 shows DSG2 promotes the adhesion of MM plasma cells to BM ECs, and is co-regulated with N cadherin. (A): BM trephine biopsies from 3 MM patients were stained for DSG2 by immunohistochemistry; shown is a representative example of a DSG2-expressing blood vessel. (B): Expression of DSG2 by the TrHBMEC cell line was assessed by flow cytometry in the parent culture (left); after sorting on DSG2 expression (centre); or after extended passage of the DSG2+ sub-culture (right). (C/D): Adhesion to a monolayer of DSG2+ TrHBMEC cells was compared for KMS11 cell lines stably expressing NT or DSG2-targeting shRNA. Following 15 minutes incubation and extensive washing, adhesion of KMS11 cells to the TrHBMEC monolayer was assessed by imaging the GFP reporter in the KMS11 cells. Shown in (C) is a summary graph of 4 independent experiments (*, p<0.05 compared to control), while (B) shows representative fluorescent images (scale bar=100 m). (E): Gene expression values for DSG2 and CDH2 were extracted from dataset GSE4581. Samples in the MS sub-group are shown in red while others (MS-negative) are shown in black. Quadrants were set visually to highlight the four distinct subsets defined by individual or co-expression of DSG2 and CDH2.

FIG. 7 shows that the viability of KMS-11 human multiple myeloma cells is reduced with overnight exposure to the proteasome inhibitor Bortezomib at 4 nM. With stable knockdown of DSG2 (via short hairpin RNA (DSG2 shRNA)), the KMS-11 cells exhibit an even further increased sensitivity to Bortezomib over 12 hours at 2 nM and 4 nM.

FIG. 8 demonstrates the detection of DSG2+ PCs in bone marrow by FACS for CD38 and DSG2 expression. In healthy controls, few cells are located in quadrant 2. In multiple myeloma patients it can be seen that there is a large number of cells in quadrant 2 cells expressing high levels of DSG2 and CD38.

FIG. 9 shows Kaplan-Meier survival estimates using flow cytometry patient data. The data directly shows that the flow cytometry test performs as expected, namely that it correctly predicts prognosis.

### DETAILED DESCRIPTION

The present disclosure relates to methods, products and systems for the prognosis of subjects suffering from multiple myeloma.

The present disclosure is based, at least in part, upon the recognition that desmoglein-2 (DSG2) is strongly up-regulated on the surface of neoplastic plasma cells in a distinct subset of multiple myeloma patients. The expression of DSG2 on neoplastic plasma cells is associated with a striking reduction in overall survival of multiple myeloma patients, thus revealing DSG2 as a novel biomarker of poor prognosis with clinical utility. In addition, it has been found that DSG2 directly contributes to adhesive interactions between multiple myeloma plasma cell and bone marrow endothelial cells, which may support the dissemination of the plasma cells to new bone marrow sites, and may be used as the basis for a screening assay for new therapeutic agents for multiple myeloma.

Accordingly, DSG2 provides a newly identified prognostic biomarker for multiple myeloma.

In particular, DSG2 can be detected in patient samples of bone marrow and/or blood, and in some antibodies can be used to validate expression levels, meaning that methods such as flow cytometry can be used for screening.

Certain embodiments of the present disclosure provide a method of prognosis for a subject suffering from multiple myeloma.

In certain embodiments, the present disclosure provides a method of prognosis for a subject suffering from multiple myeloma, the method comprising determining the level of desmoglein 2 (DSG2) in malignant plasma cells from the subject, wherein an increased level of DSG2 in the plasma cells is indicative of a poorer prognosis for the subject.

In certain embodiments, the method is used to assess or determine the response of the subject to treatment, to assess or determine clinical outcome, to asses or determine disease progression, to assess or determine survival, to screen or identify a subject suitable for treatment, and/or to select a suitable treatment regime for a subject.

Multiple myeloma (MM) is a plasma cell malignancy in which monoclonal plasma cells proliferate in bone marrow, resulting in an overabundance of monoclonal paraprotein (M protein), destruction of bone, and displacement of other hematopoietic cell lines. Malignant plasma cells in multiple myeloma are end stage antibody producing B-lymphocytes. Malignant plasma cells may be identified or identified for example by use of the CD138 marker. Methods for identifying plasma cells are known in the art, for example as described in Tellier and Nutt (2017) Eur. J. Immunol. 47: 1276-1279. Multiple Myeloma is generally described in “Multiple Myeloma” edited by M. A Gertz, S. V. Rajkumar (2013) Springer-Verlag New York Inc.

Methods for assessing whether a subject is suffering from multiple myeloma are known in the art.

In certain embodiments, the subject is a human subject. Veterinary applications of the present disclosure to animals are also contemplated.

In certain embodiments, the subject suffering from multiple myeloma is a subject suffering from stage I myeloma, stage II myeloma, or stage III myeloma, according to the Revised International Staging System (R-ISS) shown in the following table:

| Stage | Criteria |
|---|---|
| I | Serum β2 microglobulin <3.5 mg/l |
| | Serum albumin ≥3.5 g/dl |
| | Standard-risk chromosomal abnormalities |
| | (CA) |
| | Normal LDH |
| II | Not R-ISS Stage I or Stage III |
| III | Serum β2 microglobulin ≥5.5 mg/L and |
| | either |
| | High-risk CA by FISH |
| | OR |
| | High LDH |

In certain embodiments, the subject is suspected of suffering from multiple myeloma.

Methods for detecting malignant plasma cells (CD138+) are known in the art, for example as described in Flores-Montero J. et al. (2015) Clinical Cytometry 90B: 61-72.

In certain embodiments, the malignant plasma cells are obtained from bone marrow and/or blood. Methods for obtaining bone marrow samples or blood samples are known in the art. Malignant plasma cells may be detected in bone marrow samples or in peripheral blood as circulating plasma cells, for example as described in Wang J. et al (2015) Blood 126(23): 5328. Other types of samples are contemplated.

The mRNA for human desmoglein 2 is described in Genbank accession number Z26317, and is described in Arnemann et al. (1992) Genomics 13(2): 484-486. The human protein is described in UniProtKB accession number Q14126 and has the following amino acid sequence (referred to herein as SEQ ID NO. 1):

(SEQ ID NO. 1)
MARTRDRVRLLLLLICFNVGSGLHLQVLSTRNENKLLPKHPHLV
RQKRAWITAPVALREGEDLSKKNPIAKIHSDLAEERGLKITYKYTGKGI
TEPPFGIFVFNKDTGELNVTSILDREETPFFLLTGYALDARGNNVEKPL
ELRIKVLDINDNEPVFTQDVFVGSVEELSAAHTLVMKINATDADEPNTL
NSKISYRIVSLEPAYPPVFYLNKDTGEIYTTSVTLDREEHSSYTLTVEA
RDGNGEVTDKPVKQAQVQIRILDVNDNIPVVENKVLEGMVEENQVNVEV
TRIKVEDADEIGSDNWLANFTFASGNEGGYFHIETDAQTNEGIVTLIKE
VDYEEMKNLDFSVIVANKAAFHKSIRSKYKPTPIPIKVKVKNVKEGIHE
KSSVISIYVSESMDRSSKGQIIGNFQAFDEDTGLPAHARYVKLEDRDNW
ISVDSVTSEIKLAKLPDFESRYVQNGTYTVKIVAISEDYPRKTITGTVL
INVEDINDNCPTLIEPVQTICHDAEYVNVTAEDLDGHPNSGPFSFSVID
KPPGMAEKWKIARQESTSVLLQQSEKKLGRSEIQFLISDNQGFSCPEKQ
VLTLTVCEVLHGSGCREAQHDSYVGLGPAAIALMILAFLLLLLVPLLLL
MCHCGKGAKAFTPIPGTIEMLHPWNNEGAPPEDKVVPSFLPVDQGGSLV
GRNGVGGMAKEATMKGSSSASIVKGQHEMSEMDGRWEEHRSLLSGRATQ
FTGATGAIMTTETTKTARATGASRDMAGAQAAAVALNEEFLRNYFTDKA
ASYTEEDENHTAKDCLLVYSQEETESLNASIGCCSFIEGELDDRFLDDL
GLKFKTLAEVCLGQKIDINKEIEQRQKPATETSMNTASHSLCEQTMVNS
ENTYSSGSSFPVPKSLQEANAEKVTQEIVTERSVSSRQAQKVATPLPDP
MASRNVIATETSYVTGSTMPPTTVILGPSQPQSLIVTERVYAPASTLVD
QPYANEGTVVVTERVIQPHGGGSNPLEGTQHLQDVPYVMVRERESFLAP
SSGVQPTLAMPNIAVGQNVTVTERVLAPASTLQSSYQIPTENSMTARNT
TVSGAGVPGPLPDFGLEESGHSNSTITTSSTRVTKHSTVQHSYS

Orthologues and homologues of human DSG2 may be readily detected by a method known in the art, for example by using the BLAST suite of alignment tools.

In certain embodiments, the determining of the level of DSG2 in the plasma cells comprises detection of the protein and/or detection of RNA encoding the protein.

In certain embodiments, an immunological detection method is used to detect DSG2 protein. Immunological detection methods are known in the art, and include Western blotting, immunostaining, and immunoadsorption. Antibodies to DSG2 are commercially available or may be produced using a method known in the art. In certain embodiments, the detection of DSG2 utilises a mass spectrometry method. Mass spectrographic methods are known in the art.

Methods for detecting DSG2 mRNA are known in the art, and include amplification methods such as qPCR using suitable primers, in situ hybridization methods using appropriate probes, and Northern analysis using appropriate probes. The level of DSG2 mRNA may be determined, for example, with respect to the expression of the DSG2 mRNA in another cell type, such as a non-malignant plasma cell, and/or by reference to the level of another gene expressed within the malignant plasma cell, and/or a by reference to a predetermined level.

Methods for detecting DSG2 protein are known in the art. For example, human DSG2 may be detected using Western blot with a commercially available human DSG2 antibody (for example as available from R&D Systems #MAB947), using immunostaining (for example an antibody as available from R&D Systems #MAB947), and by ELISA (for example using LS Bio “Human DSG2/Desmoglein 2 ELISA Kit (Custom ELISA)” #LS-F17367). The level of the DSG2 protein may be determined, for example, with respect to the expression of the DSG2 protein in another cell type, such as a non-malignant plasma cell, and/or by reference to the level of another protein expressed within the malignant plasma cell, and/or a by reference to a predetermined level. Methods for producing antibodies, and/or an antigen binding part thereof, are known in the art.

In certain embodiments, the determining of the level of DSG2 protein comprises detecting the DSG2 by an immunological method. In certain embodiments, the determining of the level of DSG2 protein comprises detecting the DSG2 by immunohistochemistry. Methods for performing immunohistochemistry using an antibody, and/or an antigen binding part thereof, to DSG2 are known in the art.

The DSG2 protein is also expressed on the cell surface of many cells and is a functional cell surface marker. For example, DSG2 may be detected using flow cytometry. Methods for using flow cytometry are known in the art. In certain embodiments, the determining of the level of DSG2 comprises detecting cell surface expression of DSG2.

In certain embodiments, the detecting of cell surface expression of DSG2 comprises use of flow cytometry.

In certain embodiments, the level of DSG2 is based on a difference in fluorescence intensity between malignant and non-malignant plasma cells as determined by flow cytometry. In certain embodiments, the level of DSG2 is based on a difference in mean or median fluorescence intensity between plasma cells as determined by flow cytometry.

In certain embodiments, the difference in fluorescence intensity is determined by comparison to fluorescence intensity of unstained cells and/or reference cells and/or cells stained with an isotype control.

Methods for detecting RNA and protein are generally as described in Sambrook et al. Molecular Cloning: A Laboratory Manual (4th ed., Cold Spring Harbor Laboratory Press, Cold Spring Harbor, N.Y. (2012) and Ausubel et al Current Protocols in Molecular Biology (2012) John Wiley & Sons, both of which are herein incorporated by reference.

In certain embodiments, the prognosis is a poor prognosis, a good prognosis, a response to treatment, a clinical outcome, a survival rate (eg five year survival, 10 year survival rate), and/or a progression of the disease to a more severe form.

It will also be appreciated that the term “prognosis” includes within its scope a likelihood or a probability of an outcome occurring.

In certain embodiments, the prognosis is a likelihood of a response to treatment, a likelihood of a clinical outcome, a likelihood of a survival rate (eg five year survival, 10 year survival rate), and/or a likelihood of a progression of the disease to a more severe form.

In certain embodiments, an increased level of DSG2 in the plasma cells is indicative of a poorer prognosis for the subject.

In certain embodiments, an unchanged or decreased level of DSG2 in the plasma cells is indicative of a normal prognosis for the subject.

In certain embodiments, an increased level of DSG2 is indicative of a poor response of the subject to treatment, a decreased likelihood of a positive response to treatment, a reduced survival rate, or an increased likelihood of progression of the disease to a more severe state.

In certain embodiments, the level of DSG2 is determined by flow cytometry and an increased level determined by comparison with equivalent control or reference cells.

In certain embodiments, the level of DSG2 is determined by a protein detection method and/or a RNA detection method.

In certain embodiments, a level of DSG2 (protein and/or mRNA) increased by 6 fold or more, 7 fold or more, 8 fold or more, 9 fold or more, or 10 fold or more is indicative of an increased risk. In certain embodiments, a level of DSG2 increased by at least 6 fold, at least 7 fold, at least 8 fold, at least 9 fold, or at least 10 fold is indicative of an increased risk.

In certain embodiments, the genetic basis for the multiple myeloma is not known. In certain embodiments, the genetic basis for the multiple myeloma is known. In certain embodiments, the method comprises determining the genetic basis of the multiple myeloma in the subject.

In certain embodiments, the multiple myeloma comprises a genetic aberration selected from one or more of a trisomic multiple myeloma, an IgH-translocated multiple myeloma such as t(11;14), t(6;14), t(14;16), t(14;20), and t(4;14), a combined IgH-translocated/trisomic multiple myeloma, and an isolated monosomic multiple myeloma. Genetic tests for myeloma are known in the art.

In certain embodiments, the method is applicable to all genetic types of multiple myeloma.

In certain embodiments, the multiple myeloma does not have a t(4;14) translocation. In certain embodiments, the multiple myeloma has a t(4;14) translocation.

In certain embodiments, the method does not require genetic testing for multiple myeloma. In certain embodiments, the method is used in conjunction with genetic testing for multiple myeloma. In certain embodiments, the method further comprises use of one or more other biomarkers and/or clinical features, for prognosis.

In certain embodiments, the method comprises producing a prognostic score for the subject. In certain embodiments, the method comprises producing a prognostic score for the subject based on the level of DSG2 and one or more other clinical factors, such as serum β2 microglobulin level, serum albumin level, LDH level and genetic testing, which are known in the art.

In certain embodiments, the method comprises stratifying the prognostic score and assessing the subject on the basis of the stratification.

For example, a subject may be stratified into a group such as a poor prognostic group, a low survival group, an intermediate prognosis group, an intermediate survival group, or a longer term prognosis group, or a longer term survival group. Methods for stratification of scores are known in the art.

In certain embodiments, the method comprises using a computer processor means to determine the prognosis. Computer processors means are known in the art.

In certain embodiments, the computer processor means utilises software to process data associated with the level of desmoglein 2 (DSG2) in malignant plasma cells and provide a prognosis for a subject suffering from multiple myeloma based on the data. For example, the software may utilise an algorithm correlating the difference in the level of DSG2 between malignant and normal cells with a prognostic score. In another example, the software may utilise an algorithm correlating the difference in the level of DSG2 between malignant plasma cells and a control or reference value with a prognostic score. Methods for producing software to process data associated with the level of a protein or mRNA are known in the art.

In certain embodiments, the computer processor utilises software to process data associated with the level of desmoglein 2 (DSG2) in malignant plasma cells and determine the response of the subject to treatment, to determine clinical outcome, to determine disease progression, to determine survival, to identify a subject suitable for treatment, and/or to select a suitable treatment regime for the subject.

Certain embodiments of the present disclosure provide a method of assessing the response of a subject suffering from multiple myeloma to treatment.

This embodiment permits the assessment of response of a subject to the suite of different treatments that are currently used or that are developed moving forward.

In certain embodiments, the present disclosure provides a method of assessing the response of a subject suffering from multiple myeloma to treatment, the method comprising determining the level of desmoglein 2 (DSG2) in malignant plasma cells from the subject, wherein an increased level of DSG2 in the plasma cells is indicative of a reduced response of the subject to the treatment.

Methods for the determining the level of DSG2 and the correlation to response to treatment are described herein.

Examples of treatment for multiple myeloma include chemotherapy, radiotherapy, proteasome inhibitor treatment (eg bortezomib, carfilzomib, ixazomib), immunomodulatory treatment (eg thalidomide, lenalidomide, pomalidomide), antibody treatment (eg daratumumab, isatuximab, elotuzumab), stem cell therapy, and bisphoshonate therapy, all of which are known in the art. Methods for treatment of multiple myeloma are known in the art, for example as described in Lei et al (2019) Rinsho Ketsueki 60(9): 1243-1256.

In certain embodiments, the response of the subject to treatment is a poor response to treatment, a decreased likelihood of a positive response to treatment, a reduced survival rate, or an increased likelihood of progression of the disease to a more severe state.

In certain embodiments, the level of DSG2 is determined by flow cytometry and an increased level determined by comparison with equivalent control or reference cells. An increased level of DSG2 is indicative of a reduced response of the subject to the treatment.

In certain embodiments, the level of DSG2 is determined by a protein detection method and/or an RNA detection method, and an increased level of DSG2 is indicative of a reduced response of the subject to the treatment.

Certain embodiments of the present disclosure provide a method of assessing clinical outcome for a subject suffering from multiple myeloma.

In certain embodiments, the present disclosure provides a method of assessing clinical outcome for a subject suffering from multiple myeloma, the method comprising determining the level of desmoglein 2 (DSG2) in malignant plasma cells from the subject, wherein an increased level of DSG2 in the plasma cells is indicative of a poor clinical outcome for the subject.

Methods for the determining the level of DSG2 and the correlation to clinical outcome are described herein.

In certain embodiments, the clinical outcome of the subject is a poor response to treatment, a decreased likelihood of a positive response to treatment, a reduced survival rate, or an increased likelihood of progression of the disease to a more severe state.

In certain embodiments, the level of DSG2 is determined by flow cytometry and an increased level determined by comparison with equivalent control or reference cells. An increased level of DSG2 is indicative of a poor clinical outcome for the subject, is indicative of a poor response to treatment, a decreased likelihood of a positive response to treatment, a reduced survival rate, or an increased likelihood of progression of the disease to a more severe state.

In certain embodiments, the level of DSG2 is determined by a protein detection method and/or an RNA detection method, and an increased level of DSG2 is indicative of an increased level of DSG2 is indicative of a poor clinical outcome for the subject, a poor response to treatment, a decreased likelihood of a positive response to treatment, a reduced survival rate, or an increased likelihood of progression of the disease to a more severe state.

Certain embodiments of the present disclosure provide a method of assessing progression of multiple myeloma in a subject.

In certain embodiments, the present disclosure provides a method of assessing progression of multiple myeloma in a subject, the method comprising determining the level of desmoglein 2 (DSG2) in malignant plasma cells from the subject, wherein an increased level of DSG2 in the plasma cells is indicative of progression of the multiple myeloma to a more severe stage in the subject.

Methods for the determining the level of DSG2 and the correlation to progression of the multiple myeloma are described herein.

In certain embodiments, the level of DSG2 is determined by flow cytometry and an increased level determined by comparison with equivalent control or reference cells. An increased level of DSG2 is indicative of an increased rate of progression of the multiple myeloma.

In certain embodiments, the level of DSG2 is determined by a protein detection method and/or an RNA detection method, and an increased level of DSG2 is indicative of an increased level of DSG2 is indicative increased rate of progression of the multiple myeloma.

Certain embodiments of the present disclosure provide a method of assessing survival of a subject suffering from multiple myeloma.

In certain embodiments, the present disclosure provides a method of assessing survival of a subject suffering from multiple myeloma, the method comprising determining the level of desmoglein 2 (DSG2) in malignant plasma cells from the subject, wherein an increased level of DSG2 in the plasma cells is indicative of a reduced period of survival for the subject.

Methods for the determining the level of DSG2 and the correlation to survival of a subject are described herein.

In certain embodiments, the survival of the subject is a reduced five year or 10 year survival.

In certain embodiments, the level of DSG2 is determined by flow cytometry and an increased level determined by comparison with equivalent control or reference cells. An increased level of DSG2 is indicative of a reduced period of survival.

In certain embodiments, the level of DSG2 is determined by a protein detection method and/or an RNA detection method, and an increased level of DSG2 is indicative of an increased level of DSG2 is indicative of a reduced period of survival.

Certain embodiments of the present disclosure provide a method of selecting a treatment regime for a subject suffering from multiple myeloma.

In certain embodiments, the present disclosure provides a method of selecting a treatment regime for a subject suffering from multiple myeloma, the method comprising determining the level of desmoglein 2 (DSG2) in malignant plasma cells from the subject, wherein the level of DSG2 in the plasma cells is used to select a treatment regime for the subject.

Methods for the determining the level of DSG2 and the correlation to selecting a treatment regime are described herein.

In certain embodiments, an increased level of DSG2 is indicative of treatment with therapies known to be important for treating high genetic risk and/or poor prognosis myeloma. Such therapies should include proteasome inhibitors (e.g. bortezomib, carfilzomib or ixazomib) and/or monoclonal antibodies (e.g. daratumumab, isatuximab or elotuzumab) with a corticosteroid (e.g. dexamethasone or prednisolone). Immunomodulatory drugs (thalidomide, lenalidomide or pomalidomide) may be added to proteasome inhibitors and/or monoclonal antibodies and corticosteroids for increased efficacy. Methods for treating subjects using the aforementioned agents are known in the art.

In certain embodiments, a normal or decreased level of DSG2 is indicative of treatment with therapies known to be suitable for treating standard to intermediate genetic risk and/or standard to intermediate prognosis myeloma. Such therapies should include immunomodulatory drugs (thalidomide, lenalidomide or pomalidomide) and a corticosteroid (e.g. dexamethasone or prednisolone) to which proteasome inhibitors (e.g. bortezomib, carfilzomib or ixazomib) and/or monoclonal antibodies (e.g. daratumumab, isatuximab or elotuzumab) may be added to increase efficacy. Methods for treating subjects using the aforementioned agents are known in the art.

Certain embodiments of the present disclosure provide use of DSG2 as a marker.

In certain embodiments, the present disclosure provides use of desmoglein 2 (DSG2) as a marker for (i) prognosis for a subject suffering from multiple myeloma, (ii) assessing response of a subject suffering from multiple myeloma to treatment, (iii) assessing clinical outcome for a subject suffering from multiple myeloma, (iv) assessing progression of multiple myeloma in a subject, (v) assessing survival of a subject suffering from multiple myeloma, and/or (vi) selecting a treatment regime for a subject suffering from multiple myeloma.

In certain embodiments, the present disclosure provides use of DSG2 as a prognostic biomarker for multiple myeloma.

Methods for detecting DSG2 and its use as a marker as are as described herein.

In certain embodiments, the DSG2 is used as marker for detecting DSG2 RNA. In certain embodiments, the DSG2 is used as a marker for detecting DSG2 protein.

In certain embodiments, the DSG2 is used as a marker in an immunological detection method. In certain embodiments, the DSG2 is used as a marker in an immunostaining method. In certain embodiments, the DSG2 is used as a marker in an RNA expression detection method, such as RT-PCR. In certain embodiments, the DSG2 is used as a marker in a flow cytometric method.

Certain embodiments of the present disclosure provide use of an antibody to desmoglein 2 (DSG2) and/or an antigen binding part thereof for assessing multiple myeloma.

In certain embodiments, the present disclosure provides use of an antibody to desmoglein 2 (DSG2) and/or an antigen binding part thereof for (i) prognosis for a subject suffering from multiple myeloma, (ii) assessing response of a subject suffering from multiple myeloma to treatment, (iii) assessing clinical outcome for a subject suffering from multiple myeloma, (iv) assessing progression of multiple myeloma in a subject, (v) assessing survival of a subject suffering from multiple myeloma, and/or (vi) selecting a treatment regime for a subject suffering from multiple myeloma.

The term “antibody” as used herein refers to an immunoglobulin molecule with the ability to bind an antigenic region of another molecule, and includes monoclonal antibodies, polyclonal antibodies, multivalent antibodies, chimeric antibodies, multispecific antibodies, diabodies, and parts or fragments of an immunoglobulin molecule or combinations thereof that have the ability to bind to the antigenic region of another molecule with the desired affinity including a Fab, Fab′, F(ab′)₂, Fv, a single-chain antibody (scFv) or a polypeptide that contains at least a portion of an immunoglobulin (or a variant of an immunoglobulin) that is sufficient to confer specific antigen binding, such as a molecule including one or more CDRs.

Antibodies and/or antigen binding parts thereof to DSG2 are commercially available or may be produced by a method known in the art, for example as described in Ausubel et al Current Protocols in Molecular Biology (2012) John Wiley & Sons.

In certain embodiments, the antibody and/or antigen binding part thereof to DSG2 is used in an immunological detection method. In certain embodiments, the antibody and/or antigen binding part thereof to DSG2 is used in an immunostaining method. In certain embodiments, the antibody to DSG2 is used in an immunosorbent assay. In certain embodiments, the antibody to DSG2 is used in a flow cytometric method.

In certain embodiments, the antibody and/or antigen binding part thereof is used to detect DSG2 in a sample from the subject. In certain embodiments, the antibody and/or antigen binding part thereof is used to detect DSG2 in a bone marrow sample or biopsy. In certain embodiments, the antibody and/or antigen binding part thereof is used to detect DSG2 in a blood sample. Other types of samples are contemplated.

Certain embodiments of the present disclosure provide a kit for performing a method as described herein.

In certain embodiments, the kit comprises one or more reagents for detecting DSG2, assay reagents, instructions, and positive, negative and/or reference controls.

Examples of kits include flow cytometric kits, immunostaining kits, protein detection kits, RNA detection kits, or RT-PCR kits.

In certain embodiment the kit comprises an antibody to DSG2 and/or an antigen binding part thereof.

In certain embodiments, the kit comprises one or more probes, primers and/or templates for producing probes or primers for detecting or amplifying DSG2 RNA.

In certain embodiments, the kit comprises one or more further reagents for detecting markers such as CD138, CD38, CD19, and CD20, such as antibodies (or antigen binding parts thereof) which are commercially available. For example, the kit may comprise reagents for detection the aforementioned markers using flow cytometry.

In certain embodiments, the kit comprises one or more of an antibody, and/or antigen binding party thereof, for detecting CD138, an antibody, and/or antigen binding party thereof, for detecting CD38, an antibody, and/or antigen binding party thereof, for detecting CD19, and an antibody, and/or antigen binding party thereof, for detecting CD20. Certain embodiments of the present disclosure provide a method of prognosis for a subject suffering from multiple myeloma to treatment using an antibody to desmoglein 2 (DSG2) and/or an antigen binding part thereof.

In certain embodiments, the present disclosure provides a method of prognosis for a subject suffering from multiple myeloma to treatment, the method comprising using an antibody to desmoglein 2 (DSG2) and/or an antigen binding part thereof to assess the level of DSG2 in a bone marrow sample (biopsy) and/or a blood sample from the subject and thereby provide a prognosis for the subject on the basis of the level of DSG2 determined.

In certain embodiments, an increased level of DSG2 is indicative of a poorer prognosis for the subject, as described herein.

Certain embodiments of the present disclosure provide a method of assessing the response of a subject suffering from multiple myeloma to treatment using an antibody to desmoglein 2 (DSG2) and/or an antigen binding part thereof.

In certain embodiments, the present disclosure provides a method of assessing the response of a subject suffering from multiple myeloma to treatment, the method comprising using an antibody to desmoglein 2 (DSG2) and/or an antigen binding part thereof to assess the level of DSG2 in a bone marrow sample and/or a blood sample from the subject and thereby assessing the response of the subject to treatment on the basis of the level of DSG2 determined.

In certain embodiments, an increased level of DSG2 is indicative of a poor response to treatment, a decreased likelihood of a positive response to treatment, a reduced survival rate, or an increased likelihood of progression of the disease to a more severe state, as described herein.

Certain embodiments of the present disclosure provide a method of assessing clinical outcome for a subject suffering from multiple myeloma using an antibody to desmoglein 2 (DSG2) and/or an antigen binding part thereof.

In certain embodiments, the present disclosure provides a method of assessing clinical outcome for a subject suffering from multiple myeloma, the method comprising using an antibody to desmoglein 2 (DSG2) and/or an antigen binding part thereof to assess the level of DSG2 in a bone marrow sample and/or a blood sample from the subject and thereby assessing the clinical outcome for the subject on the basis of the level of DSG2 determined.

Methods for using an antibody and/or antigen binding part thereof to determine the level of DSG2, and the correlation to clinical outcome, are described herein.

In certain embodiments, an increased level of DSG2 is indicative a poor response to treatment, a decreased likelihood of a positive response to treatment, a reduced survival rate, or an increased likelihood of progression of the disease to a more severe state, as described herein.

Certain embodiments of the present disclosure provide a method of assessing progression of multiple myeloma in a subject using an antibody to desmoglein 2 (DSG2) and/or an antigen binding part thereof.

In certain embodiments, the present disclosure provides a method of assessing progression of multiple myeloma in a subject, the method comprising using an antibody to desmoglein 2 (DSG2) and/or an antigen binding part thereof to assess the level of DSG2 in a bone marrow sample and/or a blood sample from the subject and thereby assessing the progression of the multiple myeloma in the subject on the basis of the level of DSG2 determined.

Methods for using an antibody and/or antigen binding part thereof to determine the level of DSG2, and the correlation to progression, are described herein.

In certain embodiments, an increased level of DSG2 is indicative of an increased likelihood of progression of the disease to a more severe state, as described herein.

Certain embodiments of the present disclosure provide a method of assessing survival of a subject suffering from multiple myeloma to treatment using an antibody to desmoglein 2 (DSG2) and/or an antigen binding part thereof.

In certain embodiments, the present disclosure provides a method of assessing survival of a subject suffering from multiple myeloma to treatment, the method comprising using an antibody to desmoglein 2 (DSG2) and/or an antigen binding part thereof to assess the level of DSG2 in a bone marrow sample and/or a blood sample from the subject and thereby assessing the survival of the subject on the basis of the level of DSG2 determined.

Methods for using an antibody and/or antigen binding part thereof to determine the level of DSG2, and the correlation to survival, are described herein.

In certain embodiments, an increased level of DSG2 is indicative of a decreased rate of survival. In certain embodiments, an increased level of DSG2 is indicative of a reduced five year or 10 year survival rate.

Certain embodiments of the present disclosure provide a method of selecting a treatment regime for a subject suffering from multiple myeloma using an antibody to desmoglein 2 (DSG2) and/or an antigen binding part thereof.

In certain embodiments, the present disclosure provides a method of selecting a treatment regime for a subject suffering from multiple myeloma, the method comprising using an antibody to desmoglein 2 (DSG2) and/or an antigen binding part thereof to assess the level of DSG2 in a bone marrow sample and/or a blood sample from the subject and thereby selecting the treatment regime on the basis of the level of DSG2 determined.

Methods for using an antibody and/or antigen binding part thereof to determine the level of DSG2, and the correlation to treatment selection, are described herein.

In certain embodiments, an increased level of DSG2 is indicative of treatment with therapies known to be important for treating high genetic risk and/or poor prognosis myeloma. Such therapies should include proteasome inhibitors (e.g. bortezomib, carfilzomib or ixazomib) and/or monoclonal antibodies (e.g. daratumumab, isatuximab or elotuzumab) with a corticosteroid (e.g. dexamethasone or prednisolone). Immunomodulatory drugs (thalidomide, lenalidomide or pomalidomide) may be added to proteasome inhibitors and/or monoclonal antibodies and corticosteroids for increased efficacy.

In certain embodiments, a normal or decreased level of DSG2 is indicative of treatment with therapies known to be suitable for treating standard to intermediate genetic risk and/or standard to intermediate prognosis myeloma. Such therapies should include immunomodulatory drugs (thalidomide, lenalidomide or pomalidomide) and a corticosteroid (e.g. dexamethasone or prednisolone) to which proteasome inhibitors (e.g. bortezomib, carfilzomib or ixazomib) and/or monoclonal antibodies (e.g. daratumumab, isatuximab or elotuzumab) may be added to increase efficacy.

Certain embodiments of the present disclosure provide a computer-readable medium encoded with programming instructions to provide a prognosis for a subject suffering from multiple myeloma.

In certain embodiments, the present disclosure provides a computer-readable medium encoded with programming instructions executable by a computer processor means to allow the computer processor means to process data associated with the level of desmoglein 2 (DSG2) in malignant plasma cells and provide a prognosis for a subject suffering from multiple myeloma.

In certain embodiments, the programming instructions utilise an algorithm correlating the difference in the level of DSG2 with prognosis. In certain embodiments, the programming instructions utilise an algorithm correlating the difference in the level of DSG2 between malignant and normal cells with prognosis. In certain embodiments, the programming instructions utilise an algorithm correlating the difference in the level of DSG2 between malignant plasma cells and a control or reference value with prognosis.

Methods for writing programming instructions utilising algorithms are known in the art.

Certain embodiments of the present disclosure provide a computer processor comprising a computer-readable medium as described herein.

Certain embodiments of the present disclosure provide a system for providing a prognosis for a subject suffering from multiple myeloma.

In certain embodiments, the present disclosure provides a system for providing a prognosis for a subject suffering from multiple myeloma, the system comprising a computer processor having a computer-readable medium encoded with programming instructions executable by the computer processor means to allow the computer processor means to process data associated with the level of desmoglein 2 (DSG2) in malignant plasma cells from the subject and provide a prognosis for the subject.

Programming instructions are described herein.

In certain embodiments, the data is transferred over the internet to the computer processor. In certain embodiments, the data is in direct communication with the computer processor.

In certain embodiments, the system comprises a device for detecting the DSG2 and/or one or more other markers, such as a flow cytometer.

In certain embodiments, the system comprises equipment to obtain data from a sample of interest, such as a plate reader or an image acquisition device.

Certain embodiments of the present disclosure provide a method of treating a subject suffering from multiple myeloma.

In certain embodiments, the present disclosure provides a method of treating a subject suffering from multiple myeloma, the method comprising:
identifying a subject likely to be responsive to a treatment for multiple myeloma on the basis of the level of desmoglein 2 (DSG2) in malignant plasma cells; and
treating the subject so identified.

In certain embodiments, an increased level of DSG2 is indicative that the subject should be treated with therapies known to be important for treating high genetic risk and/or poor prognosis myeloma. Such therapies may include proteasome inhibitors (e.g. bortezomib, carfilzomib or ixazomib) and/or monoclonal antibodies (e.g. daratumumab, isatuximab or elotuzumab) with a corticosteroid (e.g. dexamethasone or prednisolone). Immunomodulatory drugs (thalidomide, lenalidomide or pomalidomide) may be added to proteasome inhibitors and/or monoclonal antibodies and corticosteroids for increased efficacy.

In certain embodiments, a normal level of DSG2 is indicative that the subject should be treated with therapies known to be suitable for treating standard to intermediate genetic risk and/or standard to intermediate prognosis myeloma. Such therapies should include immunomodulatory drugs (thalidomide, lenalidomide or pomalidomide) and a corticosteroid (e.g. dexamethasone or prednisolone) to which proteasome inhibitors (e.g. bortezomib, carfilzomib or ixazomib) and/or monoclonal antibodies (e.g. daratumumab, isatuximab or elotuzumab) could be added to increase efficacy.

Methods for treating subjects are known in the art, and suitable treatment regimes may be selected by a medical practitioner on the basis of the level of DSG2 determined, typically in combination with one or more clinical and/or biological characteristics of the subject and the subject's myeloma. Clinical characteristics include but are not limited to the subject's age, co-morbidities and performance status, as measured, for example, using the Eastern Cooperative Oncology Group (ECOG) scale. Biological characteristics include but are not limited to the presence and extent/severity of the myeloma defining “CRAB” criteria (hypercalcaemia, renal impairment, anaemia and bone disease), genetic risk stratification of the myeloma, and the presence or otherwise of cytopenias, neuropathy, cardiac disease, amyloidosis and plasma cell leukaemia.

Certain embodiments of the present disclosure provide a method of selecting a specific treatment for a subject suffering from multiple myeloma.

In certain embodiments, the present disclosure provides a method of selecting a specific treatment for a subject suffering from multiple myeloma, the method comprising:
determining the level of desmoglein 2 (DSG2) in malignant plasma cells from the subject; and
identifying a specific treatment for the subject on the basis of the DSG2 determined.

Clinical and/or biological characteristics of the subject and the subject's myeloma are used to select appropriate treatment. Clinical characteristics include but are not limited to the subject's age, co-morbidities and performance status, as measured, for example, using the Eastern Cooperative Oncology Group (ECOG) scale. Biological characteristics include but are not limited to the presence and extent/severity of the myeloma defining “CRAB” criteria (hypercalcaemia, renal impairment, anaemia and bone disease), genetic risk stratification of the myeloma, and the presence or otherwise of cytopenias, neuropathy, cardiac disease, amyloidosis and plasma cell leukaemia.

Certain embodiments of the present disclosure provide a method of treating a subject suffering from multiple myeloma.

In certain embodiments, the present disclosure provides a method of treating a subject suffering from multiple myeloma, the method comprising:
determining the level of desmoglein 2 (DSG2) in malignant plasma cells from the subject;
identifying a specific treatment for the subject on the basis of the DSG2 determined; and
treating the subject with the specific treatment.

In certain embodiments, an increased level of DSG2 is indicative that the subject should be treated with specific treatments known to be important for treating high genetic risk and/or poor prognosis myeloma. Such treatments include proteasome inhibitors (e.g. bortezomib, carfilzomib or ixazomib) and/or monoclonal antibodies (e.g. daratumumab, isatuximab or elotuzumab) with a corticosteroid (e.g. dexamethasone or prednisolone). Immunomodulatory drugs (thalidomide, lenalidomide or pomalidomide) may be added to proteasome inhibitors and/or monoclonal antibodies and corticosteroids for increased efficacy. Methods for using the aforementioned agents for treating a subject are known in the art.

In certain embodiments, a normal level of DSG2 is indicative that the subject should be treated with specific treatments known to be suitable for treating standard to intermediate genetic risk and/or standard to intermediate prognosis myeloma. Such treatments include immunomodulatory drugs (thalidomide, lenalidomide or pomalidomide) and a corticosteroid (e.g. dexamethasone or prednisolone) to which proteasome inhibitors (e.g. bortezomib, carfilzomib or ixazomib) and/or monoclonal antibodies (e.g. daratumumab, isatuximab or elotuzumab) may be added to increase efficacy. Methods for using the aforementioned agents for treating a subject are known in the art.

The progress and efficacy of treatment of the subject may be assessed by a medical practitioner utilising various clinical characteristics of the subject.

Certain embodiments of the present disclosure provide a method of identifying an agent for treating multiple myeloma.

In certain embodiments, the present disclosure provides a method of identifying an agent for treating multiple myeloma, the method comprising:
determining the ability of a candidate agent to reduce desmoglein 2 (DSG2) dependent adhesion of malignant plasma cells to endothelial cells; and
identifying the agent as an agent for treating multiple myeloma.

This embodiment allows the screening and identification of potential therapeutic candidates for treating multiple myeloma.

Examples of candidate agents include a drug, a small molecule, a protein, a polypeptide, a nucleic acid, a lipid, a ligand, a lipid, a carbohydrate, a nucleic acid, an oligonucleotide, a ribozyme, a biologic, an aptamer, a peptide, a cofactor, a ligand, a ligand mimetic, a receptor, an enzyme, a metal ion, a chelate, a nucleic acid, and an antibody or an antigen binding part thereof. Other types of agents are contemplated.

Methods for determining the ability of a candidate agent to reduce desmoglein 2 (DSG2) dependent adhesion of malignant plasma cells to endothelial cells are known in the art, and include in vitro and in vivo based methods.

Adhesion assays are as described herein. Flow cytometric analysis may also be used to assess DSG2 dependent adhesion. Cell adhesion methods are described for example in Kashef and Franz (2015) Developmental Biology 401(1): 165-174, hereby incorporated by reference.

In certain embodiments, the identification of a candidate agent as an agent for treating multiple myeloma utilises a suitable animal model. For example, patient-derived xenografts (PDXs) and/or cell line xenografts may be used. Suitable clinical trials can also be used to determine the efficacy of a candidate agent to treat multiple myeloma in humans.

Standard techniques may be used for cell culture, molecular biology, recombinant DNA technology, tissue culture and transfection. The foregoing techniques and other procedures may be generally performed according to conventional methods well known in the art and as described in various general and more specific references that are cited and discussed throughout the present specification. See e.g., Sambrook et al. Molecular Cloning: A Laboratory Manual (4th ed., Cold Spring Harbor Laboratory Press, Cold Spring Harbor, N.Y. (2012) and Ausubel et al Current Protocols in Molecular Biology (2012) John Wiley & Sons, both of which are herein incorporated by reference.

A listing of the various sequences described herein is provided with the file “1216533_ST25.txt”, which is herein incorporated by reference in its entirety.

The present disclosure is further described by the following examples. It is to be understood that the following description is for the purpose of describing particular embodiments only and is not intended to be limiting with respect to the above description.

### Example 1—Desmoglein 2 (DSG2) is Overexpressed in Multiple Myeloma, Regulates Adhesion to the Endothelium and is an Independent Predictor of Poor Prognosis

Abstract

Multiple myeloma (MM) is an incurable malignancy of plasma cells (PC). Although the advent of novel therapies has seen significant improvements in progression free and overall survival, patients eventually relapse and the disease is almost invariably fatal. Here, we show that the adhesion molecule desmoglein 2 (DSG2) is a novel biomarker of poor prognosis and mediates adhesion of tumor cells to the endothelium. Analysis of gene expression datasets revealed that DSG2 was overexpressed by MM PC in a subset (29-44%) of patients, but not in PC from healthy donors. Using flow cytometry and immunohistochemistry, we detected strong surface expression of DSG2 on MM PC from a similar proportion (35%) of bone marrow (BM) biopsies from newly diagnosed MM patients. Importantly, DSG2 expression was strongly predictive of poor clinical outcome, as patients with expression levels above the 70^{th} percentile exhibiting an almost 3-fold increased risk of death (HR=2.69), independent of other variables, including genetic subtype and therapy received. DSG2 was also expressed in a subset of MM cell lines, with close correlation between transcript and surface protein levels. Functional studies revealed a non-redundant role for DSG2 in mediating adhesion of MM PC to BM endothelial cells (EC). Notably, EC also express DSG2, indicating that homotypic DSG2 interactions may facilitate the entry of circulating MM PC into new sites within the BM. Together, our studies indicate that DSG2 is a biomarker for predicting disease trajectory at the time of diagnosis, and which may play a role in disease dissemination.

Introduction

Multiple myeloma (MM) is an incurable malignancy of neoplastic antibody-secreting plasma cells (PC), with a median age at diagnosis of 69 years and a median overall survival of 6-7 years (1, 2). With an age-adjusted incidence of six per 100,000 per year in the USA and Europe, it is the second most common hematological cancer (2). The past two decades has seen the introduction of novel agents that have dramatically improved overall response rates (ORR), progression-free survival (PFS) and overall survival (OS) for myeloma patients; however, disease relapse generally occurs and the disease is currently incurable.

The ability to stratify MM patients, based on the biology of their disease, is critical in guiding appropriate therapy and clinical monitoring of an individual's risk of disease progression (3, 4). For example, the t(4;14) chromosomal translocation occurs in approximately 15% of MM patients and is associated with intermediate to poor prognosis compared to patients without this translocation (5). Specifically, t(4;14)-positive MM is characterized by rapid disease progression and disease relapse, and increased tumor dissemination, reflected by an increase in the number circulating PC in the peripheral circulation (6-8). Staging systems, such as the revised international staging system (R-ISS), have been developed in order to improve treatment decisions. However, their utility in the era of an ever-increasing repertoire of novel agents to treat MM requires continual refinement to maintain prognostic validity. New appropriate biomarkers to achieve this goal are thus needed.

Desmoglein-2 (DSG2) is a surface-expressed adhesion molecule belonging to the cadherin family primarily known for its function in the formation of cell-cell adhesion multi-protein complexes known as desmosomes which are found in simple and stratified epithelia and myocardium (9, 10). In humans, four desmoglein isoforms (DSG1-4) have been identified which, together with members of the closely related desmocollin family (DSC1-3), undergo calcium-dependent homotypic and heterotypic interactions to generate the adhesive interface of desmosomes between adjacent cells. Collectively, these molecules are known as desmosomal cadherins.

Amongst the desmosomal cadherins, DSG2 appears to be unique in its ability to exist outside of desmosomes and to regulate additional biological processes (11-13). For example, an intracellular fragment of DSG2 can regulate caspase-3 cleavage and apoptosis in intestinal epithelial cells (11), while overexpression of DSG2 in suprabasal keratinocytes has been shown to induce hyperproliferation, resistance to anoikis and enhanced carcinogenesis (12). Furthermore, our studies and studies by others have demonstrated a role for DSG2 in regulating multiple aspects of endothelial cell biology, including barrier function and angiogenic activity (14, 15), and in promoting vasculogenic mimicry activity of human melanoma cells (16). These findings suggest a prominent role for DSG2 in regulating vascular function.

Intriguingly, DSG2 can also be expressed within the hematopoietic compartment, where expression is restricted to stem and progenitor populations. More specifically, expression is detectable on human hematopoietic stem/progenitor cells within adult blood, umbilical cord blood and normal bone marrow (BM), but is rapidly lost during differentiation to mature leukocyte populations (14, 17). This highlights the potential novel biological roles for DSG2, particularly considering that hematopoietic cells lack desmosomes.

Here, we demonstrate that DSG2 is strongly up-regulated on the surface of neoplastic PC in a distinct subset of MM patients. The expression of DSG2 is associated with a striking reduction in overall survival of MM patients, thus revealing DSG2 as a novel biomarker of poor prognosis with clinical utility. In addition, we show that DSG2 directly contributes to adhesive interactions between MM PC and BM endothelial cells, which may support the dissemination of MM PC to new BM sites.

Methods

Cell Lines and Culture

Human MM cell lines LP-1, KMS-11, RPMI 8226 and U266 were obtained from the American Type Culture Collection (ATCC, VA, USA); OPM2, MM.1S, MM.1R and NCI-H929 were kindly provided by Prof. Andrew Spencer (Monash University, Vic, Australia); KMS-18 were kindly provided by Prof. Junia Melo (SA Pathology, SA, Australia). MM cell lines were cultured in RPMI1640 (Gibco) supplemented with 10% FBS (HyClone) and 2 mM GlutaMax (Gibco). For semi-adherent cell lines such as KMS-11, MM.1S and MM.1R which grow as a mix of adherent and non-adherent cells, the adherent cells were collected using trypsin and the adherent and non-adherent cells pooled prior to passaging or use in experiments. The immortalized human BM endothelial cell line TrHBMEC (18) was a kind gift from B Weksler (Cornell University Medical College, NY, USA) and was cultured in HUVE medium as described (18). All cultures were periodically confirmed negative for mycoplasma using MycoAlert (Lonza).

Generation of KMS-11 Variants with Stable DSG2 Knockdown

Lentiviral vectors (pGIPZ) expressing DSG2-shRNA and non-silencing control-shRNA were obtained from Open Biosystem (Dharmacon). The following shRNA sequences were cloned into the pGIPZ expression plasmid: 5′-TGGATGTCAATGACAATAT-3′ (DSG2-shRNA60) (SEQ ID NO. 2); 5′-CCAGTGTTCTACCTAAATA-3′ (DSG2-shRNA62) (SEQ ID NO. 3); and

5′-ATCTCGCTTGGGCGAGAGTAAG-3′ (non-silencing shRNA) (SEQ ID NO. 4). Replication incompetent lentiviral particles were generated by transiently co-transfecting HEK293T cells with ViraPower Lentiviral Support Kit (Invitrogen) and pGIPZ-shRNA vectors using Lipofectamine 2000 (Invitrogen). Lentiviral supernatant was harvested 72 hours post-transfection and used to transduce 1×10⁵ KMS-11 cells that were seeded in a 6-well plate, in the presence of 4 μg/mL polybrene. The plate was centrifuged at 1,800 rpm for 30 minutes at 37° C. and the transduced cells were selected by the addition of 1 μg/mL puromycin to cultures 72 hours later. Puromycin was continually added to maintain culture of cells with the transduced vectors and DSG2 expression was routinely checked using flow cytometry and western blot.

Patient Samples

Peripheral blood (PB) and posterior superior iliac spine BM aspirates and trephine biopsies were collected from 17 newly diagnosed MM patients, as defined by standard diagnostic criteria (19) (median age: 64 years [range 41-81]; male:female, 8:9).

Flow Cytometry

For patient samples, cell surface expression of DSG2 was assessed by flow cytometry on viable CD38++ CD138+CD45loCD19− MM PC, as previously described (20). Briefly, BM and PB mononuclear cells were stained with anti-DSG2 antibody (clone 6D8, Invitrogen) or no primary antibody [fluorescence minus one (FMO) control] followed by a PE-goat anti-mouse IgG secondary antibody (Southern Biotech) prior to staining with antibodies CD38-PE-Cy7 (HIT2; BioLegend), CD138-AlexaFluor-647 (B-B4; Serotec), CD45-FITC (J.33; Beckman Coulter), CD19-Brilliant Violet 421 (HIB19; BioLegend) and the viability dye hydroxystilbamidine (FluoroGold; Invitrogen, Life Technologies). For analysis of DSG2 expression on MM cell lines, cells were incubated with Alexa Fluor 488-conjugated anti-DSG2 monoclonal antibody (clone CSTEM28; Life Technologies) as per the manufacturer's instructions. Samples were analyzed on an LSRFortessa flow cytometer (BD Biosciences).

Immunohistochemistry

Immunohistochemistry was performed using the ADVANCE™ HRP polymer system kit (Dako). Formalin fixed, decalcified, paraffin embedded (FFPE) trephine biopsies were sectioned, dewaxed and subjected to heat-mediated antigen retrieval (20 mins in a microwave) in pH 6.0 citrate buffer. After cooling, sections were treated with the endogenous peroxidase block provided in the kit for 10 min at RT, rinsed and then incubated for 30 min at RT with primary mAb against DSG2 (clone #141409, R&D Systems, 0.9 μg/ml final concentration) or CD138 (clone MI15; Dako, 1:100 dilution from stock) or an isotype-matched (IgG1) control antibody (Abcam, 0.5 μg/ml). Detection steps were performed according to the manufacturer's recommendations, followed by reaction with DAB, counterstaining using Mayer's hematoxylin and mounting in DPX.

Adhesion Assay

BMEC were seeded in 2.5 ml of HUVE media onto 35 mm×10 mm culture dishes (Corning) until a confluent monolayer was generated. KMS-11 cells (1×10⁶) transduced with the different shRNAs were added onto the monolayer in 1 ml of HBSS. Following a 15-minute incubation at 37° C. and 5% CO₂, the HBSS in the dishes was aspirated, and each dish was rinsed twice with 2 ml of HBSS to remove unbound cells. A third wash was performed with the dishes containing 2 ml of HBSS placed on an orbital mixer (Ratek) at a speed setting of 5. Any unbound cells were aspirated and 1 ml of fresh HBSS was added to the dishes. Avoiding the edges, fluorescent images were taken at 7-8 fields of views across the middle of the dish under a 10×/0.30 objective on an IX73 inverted fluorescent microscope (Olympus) using the cellSens Dimension software (Olympus). The number of bound fluorescent cells was quantified using ImageJ (NIH).

Inverse Invasion Assay

Inverse invasion assays were performed as previously described (21). Briefly, BD Matrigel Basement Membrane Matrix (BD Bioscience, concentration approx. 9 mg/ml) was mixed 1:1 with PBS and allowed to polymerize in 8 μm pore Transwell inserts (Corning) for at least 1 hour at 37° C. Inserts were then inverted, and 10×10⁴ cells (which had been serum starved overnight in 0% FBS) were seeded directly onto the outside surface of the filter and incubated at 37° C. for 4 hours to allow cells to adhere, before being returned to the culture well in the correct orientation. Serum-free medium was added to the well, and medium containing 20% FBS was added into the upper chamber on top of the Matrigel to create a chemotactic gradient. Ninety six hours after seeding, invading cells were stained with propidium iodide (Invitrogen) for 30 minutes. Serial optical sections were captured at 20 μm intervals. The fluorescence intensity for each section was measured using Image J plugin Area Calculator.

Lysate Preparation and Western Immunoblotting

KMS-11 cells cultured on 100 mm Petri dishes were washed twice with ice-cold PBS and solubilized in RIPA lysis buffer (150 mM NaCl, 50 mM Tris-HCl, pH 7.5, 1% Triton X-100, 1% deoxycholate, 0.1% SDS, and 2 mM EDTA) containing both protease (cOmplete™, Roche) and phosphatase inhibitors (PhosStop™, Roche) for 10 min on ice. The lysates were clarified by centrifugation at 13,000×g for 10 min at 4° C., and protein concentrations determined using the bicinchoninic acid protein assay kit (Pierce). Equal concentrations of soluble lysate were boiled in reducing SDS sample buffer (10 mM Tris-HCl, pH 8.0, 1% SDS, 10% glycerol, 20 nM DTT and bromophenol blue) for 5 min. Samples (50 μg/lane) were resolved in 4-12% Bis-Tris polyacrylamide gels (BioRad) and electrophoretically transferred to nitrocellulose filters (Pall Corporation). The filters were blocked with blocking buffer (Odyssey Blocking Buffer, Li-COR) overnight at 4° C. and then incubated for 1 h with either the rabbit anti-Carboxy terminal DSG2 (Bethyl Laboratories), rabbit anti-phospho p44/42 MAPK (ERK1/2, Thr202/Tyr204; CST), rabbit anti-phospho AKT (Ser473; CST) and mouse anti-IKBα (CST) at 1:1000 dilution in blocking buffer. After extensive washing with several changes of TTBS, the filters were incubated for 45 min with either IRDye 800CW goat anti-rabbit IgG (Li-COR) or IRDye 680CW goat anti-mouse at a 1:10,000 dilution in blocking buffer. After washing, immunoreactivity was detected by using the Odyssey infrared imager (Li-COR). To normalize protein levels, filters were then stripped with 2% SDS, 100 mM 0-mercaptoethanol in 62.5 mM Tris-HCl, pH 6.8, for 30 min at 70° C., and re-probed with either rabbit anti-GAPDH, anti-p44/42 MAPK (Erk1/2) or anti-AKT (CST) as described above. Band intensities were quantitated by densitometry using the software associated with the Odyssey infrared imager (Li-COR).

Analysis of DNA Microarray and RNAseq Datasets

DSG2 gene expression was assessed in CD138-selected human BM PC from patients with newly diagnosed MM, monoclonal gammopathy of undetermined significance (MGUS) and healthy controls in publically available microarray datasets E-GEOD-16122 (normal, n=5; MGUS, n=11; MM, n=133; PCL, n=9) (22) and E-MTAB-363 (normal, n=5; MGUS, n=5; MM, n=155) (23). Data was processed and analyzed as previously described (24). A publically available dataset comprising RNAseq data from a panel of 65 human myeloma cell lines (25) was downloaded from www.keatslab.org and used to assess DSG2 gene expression in human MM cell lines. Dataset GSE4581 was used to assess the link between DSG2 expression and overall survival, and to perform differential gene expression analyses (26). Data were downloaded in R with the aid of the GEOquery library (27) and log 2 transformed. The transformed data were analyzed in Bioconductor using limma library (28) to perform differential gene analysis and pHeatmap library to generate gene expression heatmaps. Individual samples were assigned to subsets (MS, CD1, CD2, LB, HY, MF or PR) according to labels provided by the data owner, as described previously (26).

Statistical Analyses

Survival analyses based on DSG2 subgroups (high versus low on MM PC in bone marrow) were performed using Stata version 14 (StataCorp, College Station, TX), using the Kaplan-Meier method with log-rank tests to assess differences in survival between groups. A multivariate Cox proportional hazards regression model was then constructed to estimate the risk of dying based on DSG2 expression level, therapy administered, and MS+ versus MS− groupings at diagnosis. Other statistical analyses (contingency analysis using Fisher's Exact test, Spearman's correlation analysis and Mann-Whitney test) were performed in GraphPad Prism v5.04. Test statistics resulting in a p-value less than 0.05 were deemed significant.

Results

DSG2 is Expressed by MM PC at the Gene and Protein Level in a Distinct Subset of MM Patients

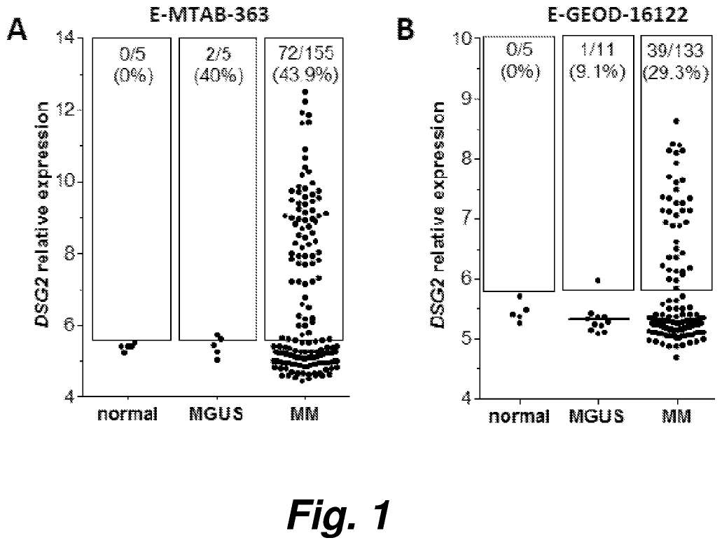

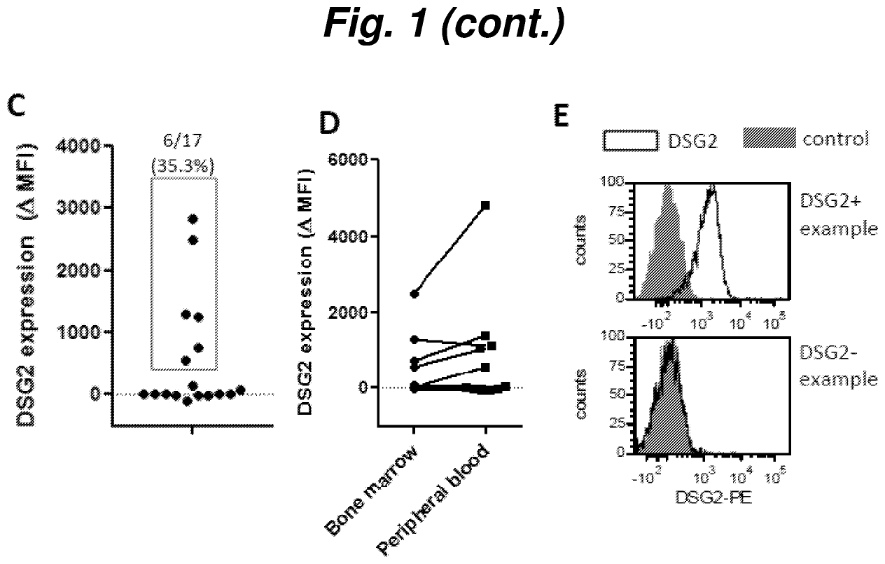

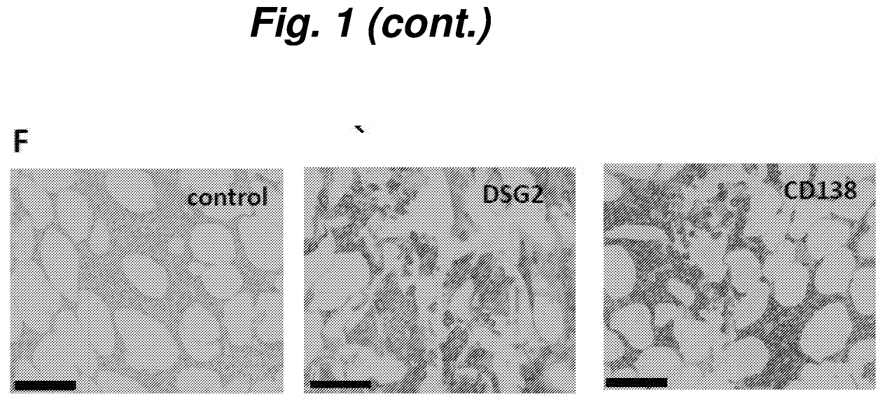

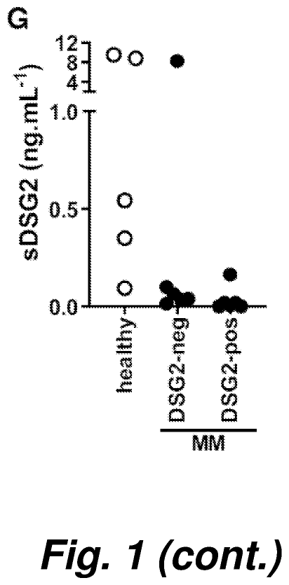

To assess the expression of DSG2 in MM PC, we analyzed gene expression data from two publically available DNA microarray datasets: E-GEOD-16122 (22) and E-MTAB-363 (23). Both of these studies measured gene expression within CD138⁺ BM PC from newly diagnosed MM patients and compared these with PC from normal BM or MGUS patients. As shown in FIG. 1 (A-B), DSG2 was expressed by MM PC in a distinct subset of MM patients. Threshold values for DSG2 expression were established for each dataset based on mean+2SD of the normal controls, and the proportion of DSG2+ samples above this threshold was determined. For both datasets, 0/5 normal BM PC samples were classified as DSG2+. In contrast, 72/155 of MM patient samples (46.5%) were DSG2+ in the E-MTAB-363 dataset and 39/133 (29.3%) samples were DSG2+ in the E-GEOD-16122 dataset. Marginal expression of DSG2 was noted in 2/5 (40%) MGUS patients in E-MTAB-363 and 1/11 (9.1%) MGUS patients in E-GEOD-16122, although the significance of this observation was unclear. A contingency analysis performed on the pooled data revealed a statistically significant difference in the proportion of individuals with DSG2+ PC between normal donors and MM patients (Fisher's Exact test; p<0.05). Interestingly, no other members of the desmosomal cadherin gene family (DSG1, DSG3, DSC1, DSC2 or DSC3) were overexpressed in MM PC in either study (data not shown). This suggests a potential function for DSG2 in these cells that may be unrelated to its canonical function in desmosome formation.

To assess whether DSG2 is also expressed as a surface protein by MM PC, patient BM mononuclear cells were assessed for DSG2 expression by multi-color flow cytometry. MM PC were gated according to a CD38++ CD138+ CD45lo CD19− phenotype and DSG2 expression was quantified as the difference in median fluorescence intensity (AMFI) between the DSG2-stained sample and fluorescence-minus-one (FMO) control (FIG. 1C-E). In keeping with the analyses of gene expression, we found that DSG2 surface protein was expressed by MM PC in the BM of distinct subset of patients. Of the 17 BM samples analyzed, 6 were DSG2+ (35.3%), a proportion very similar to the gene expression analyses (29.3-43.9% DSG2+, depending on the dataset analyzed), thus highlighting a close relationship between gene and protein expression.

For 11 of these patients, a population of MM PC was readily detectable in the peripheral blood as well as the BM. As such, we also analyzed peripheral blood (PB) mononuclear cell samples from these patients to assess DSG2 expression within the circulating CD38++ CD138+ CD45lo CD19− (MM PC) population. Notably, there was close association between DSG2 expression on PC from both the BM and PB (FIG. 1D). Thus, for all patients whose MM PC in the BM were DSG2+, their MM PC in the PB were also DSG2+. In contrast, the majority of patients whose MM PC in the BM lacked DSG2, were similarly DSG2− in the PB. Interestingly, however, one patient had MM PC in the PB which were DSG2+, while their BM counterparts were DSG2−.

Finally, for three of the patient samples analyzed by flow cytometry (two DSG2+ and one DSG2−), BM trephine biopsies were also available for analysis of DSG2 expression by immunohistochemistry (FIG. 1F). This analysis confirmed homogenous membrane expression of DSG2 by MM PC for the two patients who were also positive for DSG2 by flow cytometry. As expected, the patient for whom DSG2 was undetectable by flow cytometry also lacked membrane DSG2 staining by immunohistochemistry.

An extracellular fragment of SCG2 can be shed from the surface by MMP9 and ADAM17. To examine whether soluble DSG2 is detectable in patients with MM, and ELISA was used to test the serum of 13 MM patients previously identified in FIG. 1C to be either negative or positive for DSG2 on their MM PCs as well from serum from healthy donors. FIG. 1G shows that sDSG2 is detectable in a proportion of the donors. However, no discernable increase in DSG2 was identified for the DSG2⁺ MM patients.

Together, these analyses demonstrate that DSG2 expression is induced in malignant PC in a distinct subset of MM patients. Furthermore, expression can be detected using multiple approaches (measurement of gene expression in BM specimens; flow cytometric analysis of either BM or peripheral blood; and IHC analysis of diagnostic trephine biopsies), with largely concordant results.

DSG2 is Expressed by a Distinct Subset of Human MM Cell Lines

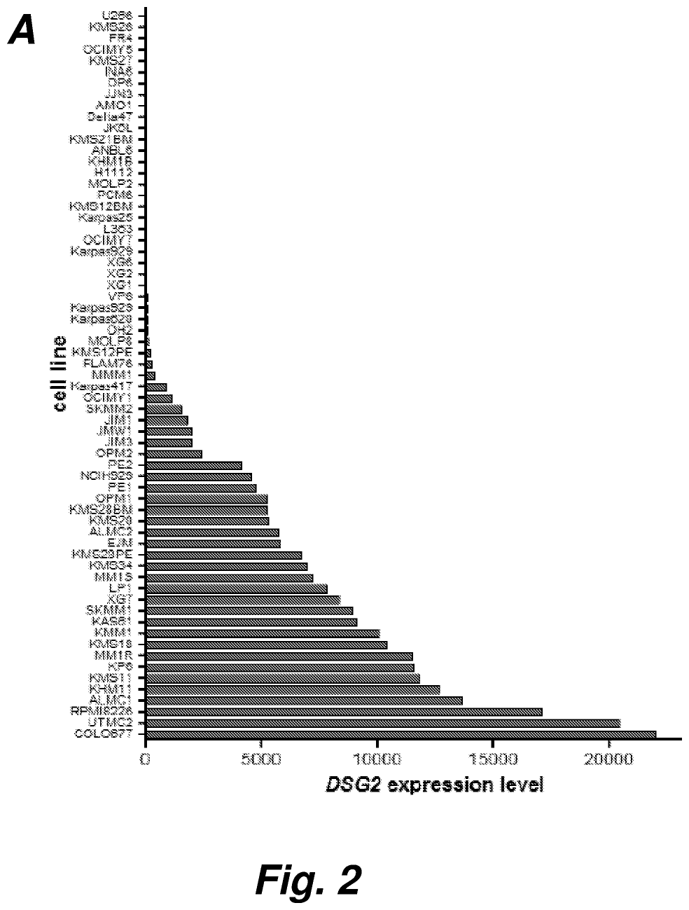

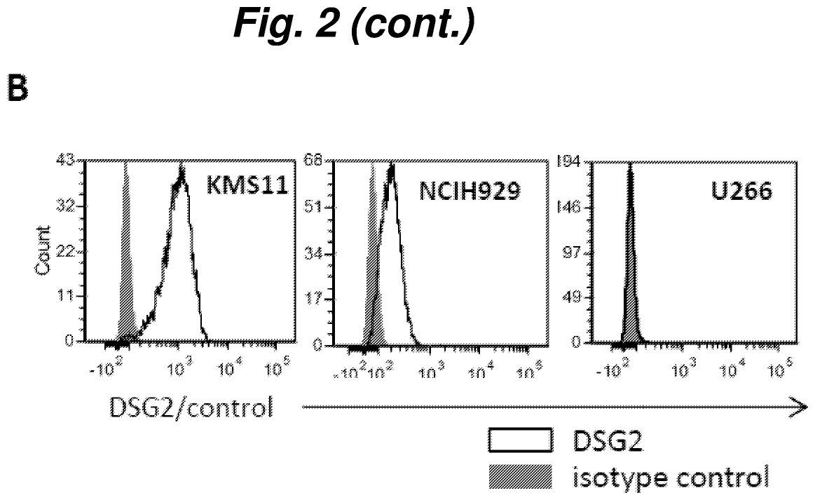

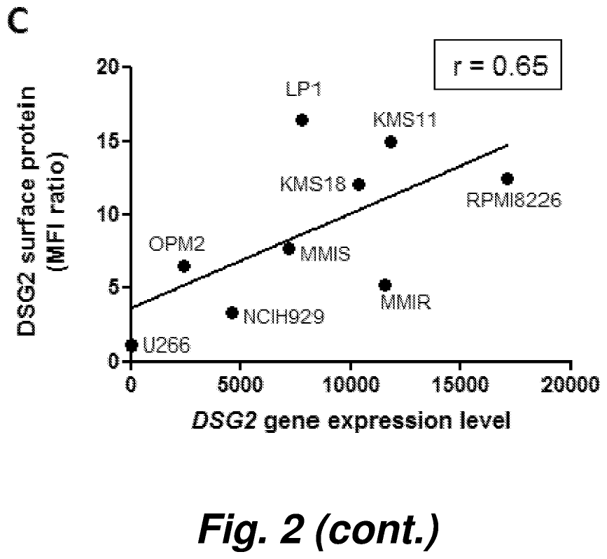

To extend our analyses of ex vivo patient samples, we also investigated DSG2 expression in patient-derived MM cell lines. Initially, gene expression was assessed in a panel of 65 human MM cell lines by interrogating publicly available RNA sequencing data (25) (FIG. 2A). Similar to the patient samples, more than half (55.4%) of the human MM cell lines tested (using an expression threshold of 100) also expressed DSG2. For nine of these cell lines, we also measured expression of DSG2 surface protein by flow cytometry (FIG. 2 B-C). DSG2 surface protein was readily detectable on cells which expressed DSG2 mRNA (e.g. lines KMS-11 and NCI-H929 as shown in FIG. 2B left and center) but was undetectable in the U266 line which had gene expression below the expression threshold (FIG. 2B, right). Moreover, as shown in FIG. 2C, there was a clear positive correlation between levels of gene expression and levels of surface protein, (Spearman's correlation coefficient r=0.65).

DSG2 Expression is an Independent Predictor of Poor Survival Despite Association with NSD2 Expression

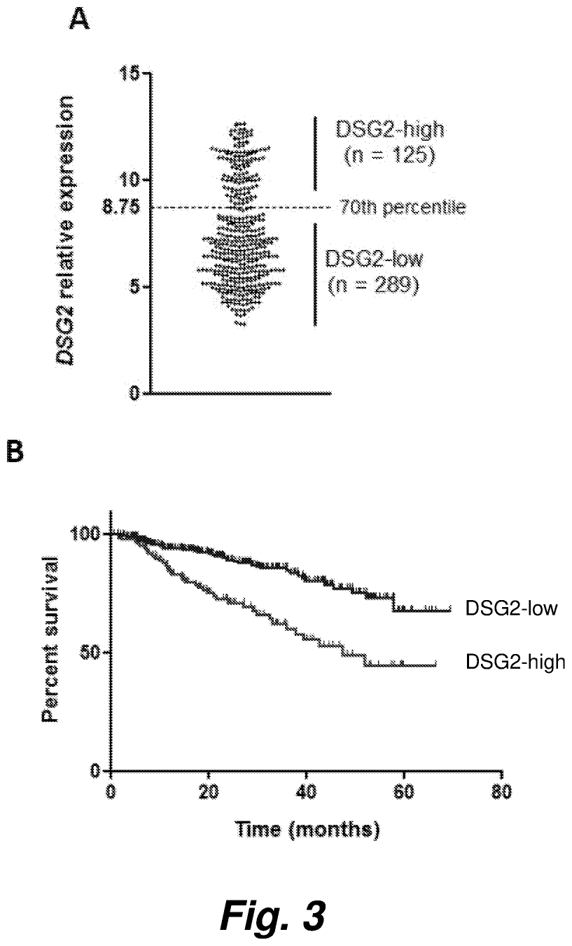

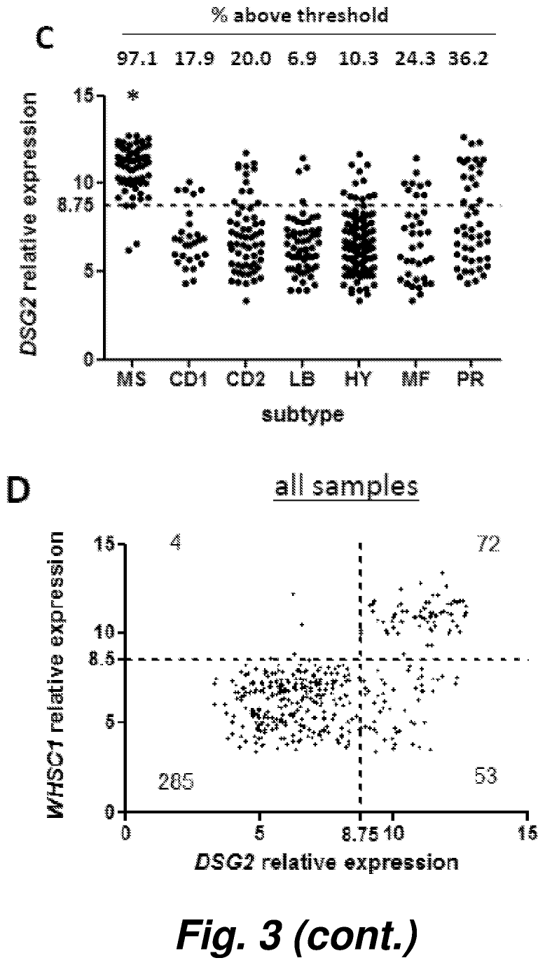

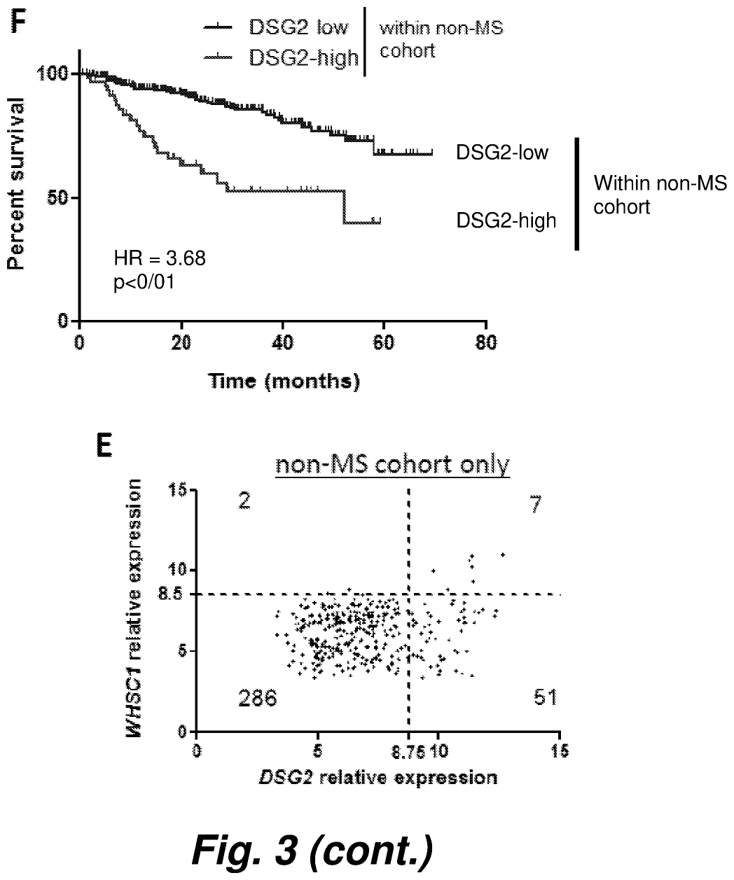

To assess a potential link between DSG2 expression and overall survival of MM patients, we analyzed the publically available gene expression dataset GSE4581, in which CD138⁺ MM PC were purified from the BM of newly-diagnosed MM patients using magnetic sorting, and gene expression was subsequently analyzed using cDNA microarray (26). Analysis of DSG2 gene expression in this dataset revealed a clear separation of samples into DSG2-high (n=125) and DSG2-low (n=289) groups, with a division based on the 70^{th} percentile of DSG2 expression (FIG. 3A). Of note, when these groups were compared by Kaplan-Meier survival analysis, a markedly inferior overall survival was observed for patients with high DSG2 expression compared to those with low DSG2 expression (p<0.001, FIG. 3B). The median overall survival (OS) in the DSG2-high group was 47 months whilst the median OS was not reached in the DSG2-low group. Notably, the risk of death was 2.7 times higher in the DSG2-high group (HR 2.69, 95% CI 1.73-4.18, p<0.001).

The t(4;14)(q13;q32) translocation is a relatively common genetic event in MM (˜15%) (REF), resulting in overexpression of the histone methyltransferase NSD2 (also known as MMSET or WHSC1), resulting from fusion between NSD2 and the IGH locus (4). As the t(4;14) translocation is an established genetic marker of intermediate to poor prognosis (5), as is the expression of NSD2 (26), we hypothesized that there may be a link between DSG2 expression and reduced survival due to its association with NSD2 expression. To address this possibility, we performed further analysis of the GSE4581 dataset. In this dataset, patient samples have been allocated to one of seven subgroups based on gene expression signatures by the original study authors (26). As shown in FIG. 3C, DSG2 expression was significantly higher in the MS subgroup compared to each of the other subgroups (Kruskal-Wallis test; p<0.05). Moreover, patients in the MS subgroup were almost uniformly DSG2-high (66/68 patients; 97.1%), using the same threshold for expression as used for the full cohort analysis. Importantly though, each of the other six subgroups also harbored a subset of DSG2-high samples, ranging from 6.9% to 36.2% of the patients (FIG. 3C), and DSG2 retained overall prognostic significance after adjusting for all MM genetic subgroups concurrently (HR 3.03, 95% CI 1.75-5.25, p<0.001). Even in patients with hyperdiploidy, which occurs in up to 50% of MM and is associated with a more favorable prognosis (4), DSG2-high MM PC expression identifies a subgroup with notably poorer survival (HR 3.21, 95% CI 1.04-9.92, p=0.04). Moreover, high DSG2 expression identifies a poor-prognosis subset of patients in 2 of 4 favorable prognosis genetic subgroups and in the MF (poor prognosis) subgroup, characterized by MAF rearrangements. The effect of DSG2 expression on patient survival in each genetic subgroup is shown in Table 1. Taken together, DSG2 appears to be a strong predictor of poorer patient survival, independent of cytogenetic risk group.

TABLE 1
The effect DSG2 expression on patient survival in each genetic subgroup

| | | | | | 95% | |
|---|---|---|---|---|---|---|
| | Genetic | Prognostic | DSG2 | Hazard | Confidence | |
| | Grouping | Significance | (high/low) | Ratio | Interval | p value |
| CD-1 | Cyclin D1 | Low-risk | 5/23 | 6.60 | 0.91-47.56 | 0.06 |
| (n = 28) | and cyclin | | | | | |
| | D3: | | | | | |
| | t(11; 14) and | | | | | |
| | t(6; 14) | | | | | |
| CD-2 | Cyclin D1 | Low-risk | 12/48 | 0.62 | 0.07-5.18 | 0.66 |
| (n = 59) | and cyclin | | | | | |
| | D3: | | | | | |
| | t(11; 14) and | | | | | |
| | t(6; 14) | | | | | |
| HY | Hyperdiploidy | Low-risk | 12/104 | 3.21 | 1.04-9.92 | 0.04 |
| (n = 116) | | | | | | |
| LB | Low bone | Low-risk | 4/54 | 9.08 | 1.65-50.13 | 0.01 |
| (n = 58) | disease | | | | | |
| MF | MAF: | High-risk | 9/28 | 8.76 | 1.70-45.20 | 0.01 |
| (n = 37) | t(14; 16) and | | | | | |
| | t(14; 20) | | | | | |
| MS | MMSET: | High-risk | 66/2 | Indeterminate | Indeterminate | Indeterminate |
| (n = 68) | t(4; 14) | | | | | |
| PR | Proliferation | High-risk | 17/30 | 2.00 | 0.79-5.11 | 0.15 |
| (n = 47) | related | | | | | |

The detection of DSG2 in non-MS subsets suggests that expression of this gene may arise through alternate mechanisms that are independent of NSD2. However, the possibility remained that some samples in these other non-MS subsets expressed NSD2 despite not being classified as MS subtype according to the multi-gene signatures, and that NSD2 was driving DSG2 expression in these samples as well. To investigate this, we plotted expression values for DSG2 against those for NSD2. When all samples were included in the analysis, a clear subset co-expressed both genes at high levels (upper right quadrant in FIG. 3D)). On this basis, a threshold for significant expression of NSD2 was set at 8.5. Unsurprisingly, when this same threshold was applied specifically to the non-MS samples (FIG. 3E), the vast majority (337/346; 97.4%) fell below the threshold for NSD2 expression. More importantly, this was also true specifically within the DSG2-high subset, where 51/58 (87.9%) of DSG2-high samples lacked significant co-expression of NSD2. These data suggest that NSD2 is not the only factor that drives DSG2 expression in MM PC.

We then repeated the Kaplan-Meier survival analysis specifically for the non-MS patients. When these samples were stratified into DSG2-high and DSG2-low groupings, the DSG2-high group again had significantly poorer survival (p<0.001, FIG. 3F). Moreover, survival differences between the DSG2-high and DSG2-low groupings were even more disparate in this analysis of non-MS patients than in the analysis of the full cohort shown in FIG. 3B. The median OS for the DSG2-high group was 52 months, whilst the median OS was not reached for the DSG2-low group. Although the median overall survival time for the DSG2-high group in this non-MS cohort analysis was longer than that of the DSG2-high group for the entire cohort, a greater reduction in the proportion of surviving DSG2-high MM patients was evident in the first two years in the non-MS analysis, suggesting an earlier adverse effect of DSG2 on survival in this subgroup of patients (FIG. 3F). Moreover, non-MS patients who were categorized as DSG2-high had an almost four-fold greater risk of dying compared to those who were categorized as DSG2-low (HR 3.68, 95% CI 2.18-6.22, p<0.001).

Finally, whilst the therapy patients received (total therapy 2 or 3) had no impact on overall survival, the predictive ability of DSG2 was even greater after concurrently adjusting for both MS subset and therapy administered (HR 4.30, 95% CI 2.47-7.48, P<0.001). Together, these findings clearly suggest that DSG2 is predictive of the overall survival of newly-diagnosed MM patients, independent of NSD2 expression and thus, by extension, the t(4;14) translocation. Furthermore, the type of treatment administered does not abrogate the poorer prognosis of high MM-PC DSG2 expression.

Analysis of Genes Differentially Expressed Between DSG2+ and DSG2-MM PC

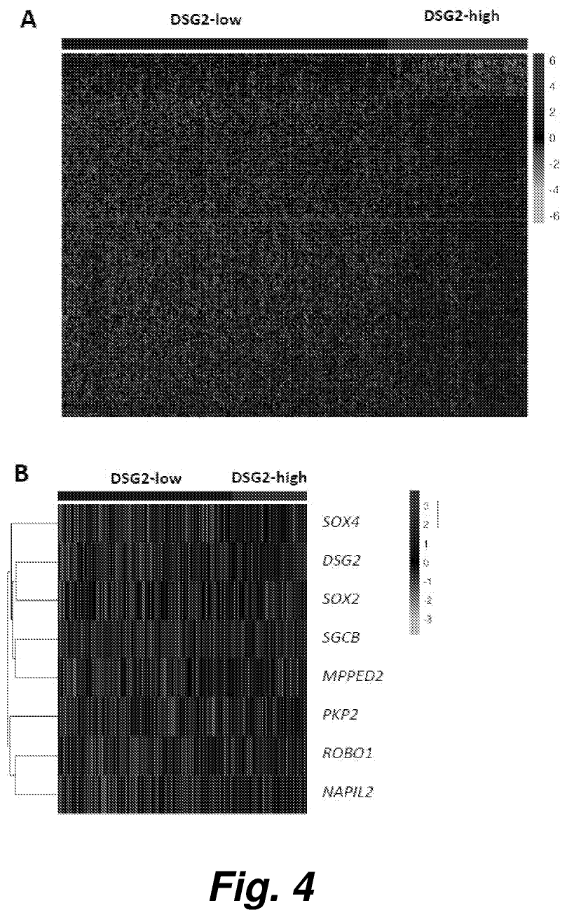

We next compared global gene expression profiles in patient samples defined in FIG. 3 as DSG2-high or DSG2-low. When analyzing the entire patient cohort, the DSG2-high and DSG2-low subsets revealed highly divergent gene expression profiles, with 316 significantly differentially expressed genes (FIG. 4A). However, these distinct transcriptional profiles may be driven largely by the t(4;14) translocation and subsequent expression of the NSD2 methyltransferase which is known to regulate hundreds of genes (29). We therefore also analyzed the non-MS samples separately which revealed a strikingly different pattern, with only seven genes (excluding DSG2) differentially expressed between the DSG2-high and DSG2-low subsets (FIG. 4B). Thus, DSG2 expression in MM appears to arise by two distinct means; either as part of a wider genetic program induced by NSD2, or as an isolated event induced by unknown mechanisms and not associated with consistent co-regulation of a large set of other genes.

No Detectable Role for DSG2 in Regulating the Growth, Survival, Migration or Major Signaling Pathways of the KMS-11 MM Cell Line

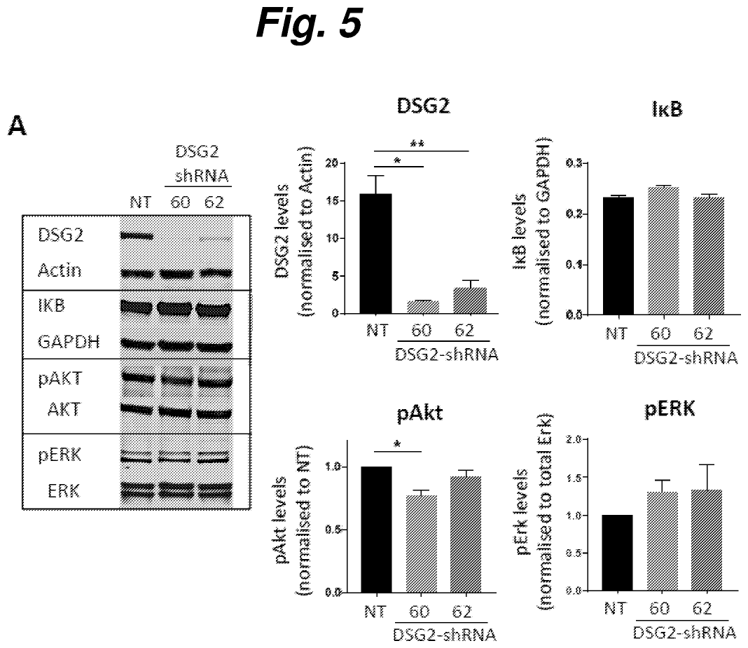

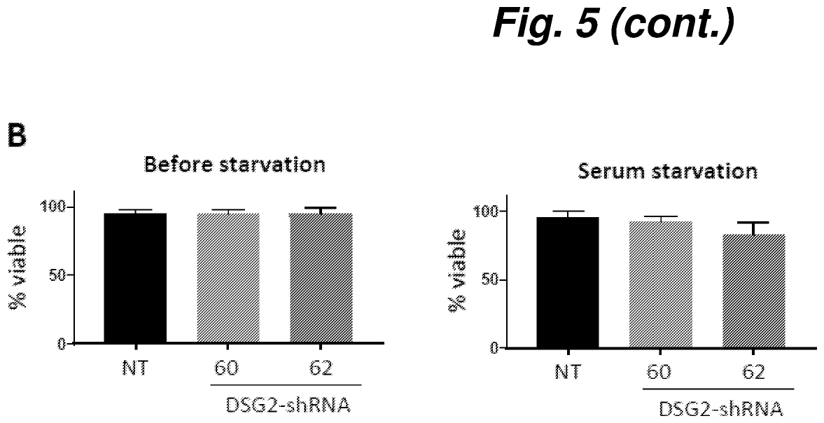

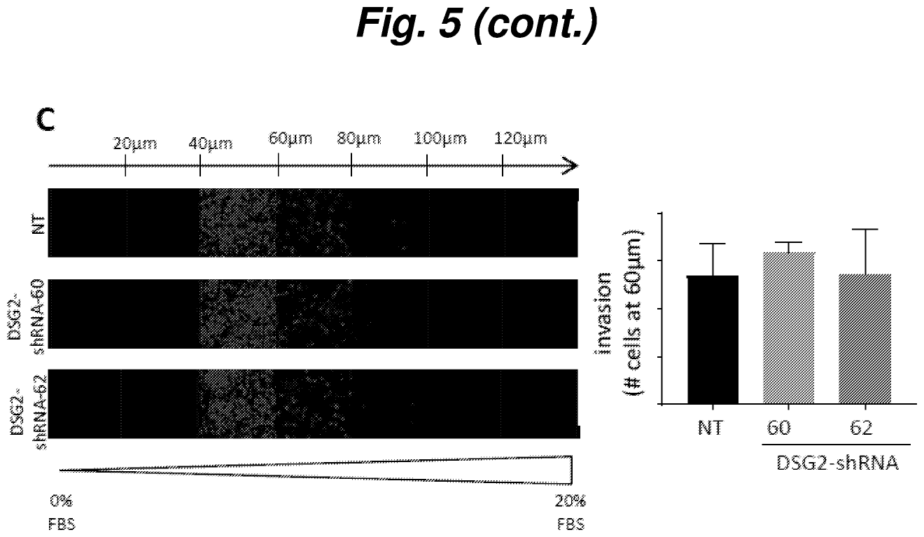

To begin exploring potential biological functions for DSG2 in MM PC, we used the DSG2+ KMS-11 cell line (FIG. 2B) to stably knockdown DSG2 expression by shRNA. Efficient knockdown of DSG2 protein was achieved using two different DSG2-targeting shRNA constructs (60 and 62), as confirmed by Western blot (FIG. 5A). These two DSG2-KD cell lines were maintained in culture alongside cells expressing a non-targeting control shRNA (NT), with no noticeable differences in growth rate or morphology between the three lines. In keeping with this observation, when DSG2-KD cells were compared to NT control cells for activation of major growth and survival signaling pathways by Western blot analysis (FIG. 5A), no significant differences were noted in the expression of IκB or the phosphorylation of ERK. While a marginal reduction in phosphorylation of AKT was noted with one DSG2-targeting shRNA, this was not observed with the other construct. Cell viability was compared both under normal culture conditions in 10% serum and after 24 hours of serum starvation, with no significant differences between cell lines expressing or lacking DSG2 (FIG. 5B). Finally, inverse invasion assays (adapted from (31)) were used to compare migration and invasion capacity, and again no difference was noted between the cell lines (FIG. 5C). Thus, in contrast to previous studies examining the role of DSG2 in several other cancerous and normal cell types (11, 12, 32, 33), DSG2 did not regulate the growth, survival or migration of MM PC, at least under the conditions tested.

DSG2 is Expressed by Endothelial Cells within the BM and Mediates Adhesive Interactions with MM PC

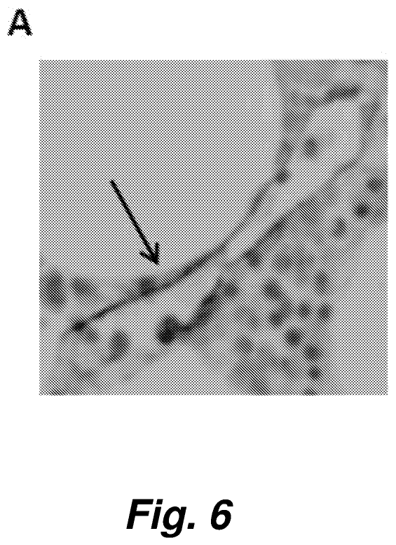

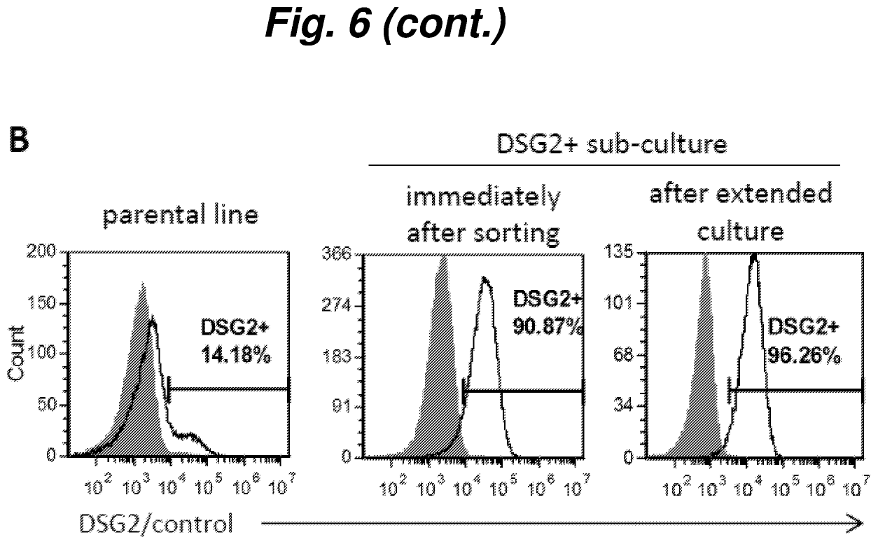

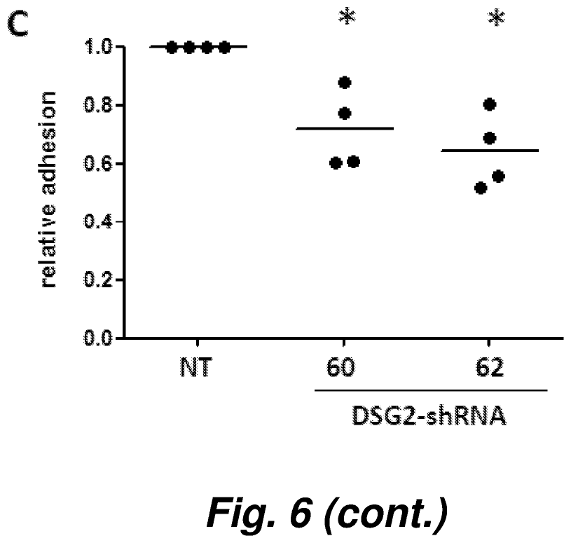

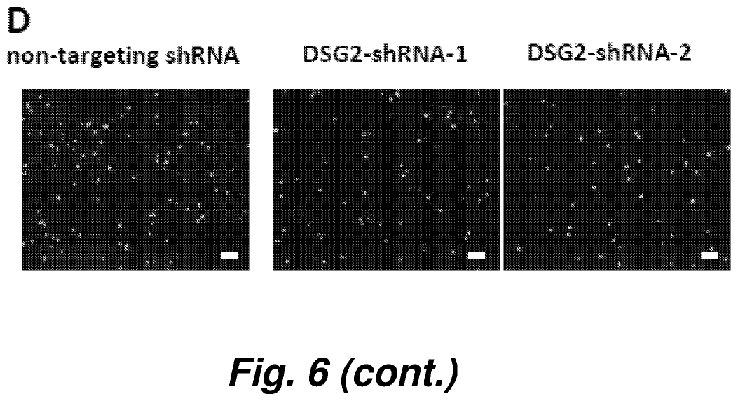

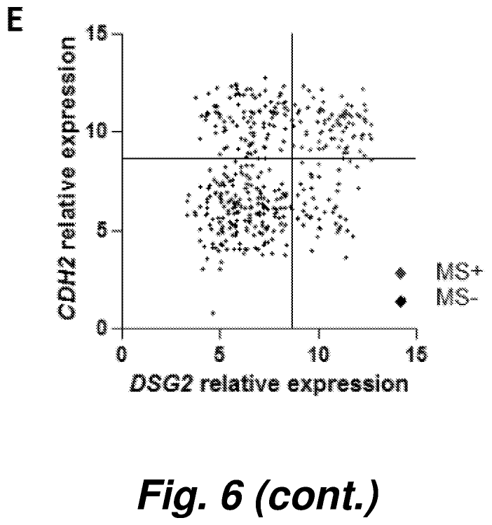

While analyzing the BM trephine biopsies stained for DSG2 shown in FIG. 1F, we noted that expression of DSG2 was not limited to the MM PC but was also frequently detectable on blood vessel structures in all three of the BM specimens examined. An example of a DSG2-expressing blood vessel is shown in FIG. 6A. To further analyze expression of DSG2 by BM endothelial cells, we used flow cytometry to measure DSG2 expression on an immortalized endothelial cell line derived from human BM (TrHBMEC)(18) and identified a distinct sub-population of DSG2+ cells (FIG. 6B; left). This DSG2+ subset was enriched by FACS and expanded, producing a subculture of TrHBMEC that expressed DSG2 uniformly for further analysis (FIG. 6B; center and right) (referred to as BMEC-DSG2).

Given that DSG2 can mediate cell-cell adhesion via homotypic interactions with other DSG2-expressing cells, we hypothesized that DSG2 expression by MM PC may facilitate binding to DSG2-expressing endothelial cells in the BM. To test this, KMS-11 cells with or without DSG2 knockdown were incubated with confluent monolayers of BMEC-DSG2 cells. Non-bound KMS-11 cells were removed by extensive washing and those which remained adhered to the BMEC-DSG2 monolayer were quantified via their GFP tag (encoded within the shRNA construct). Notably, both of the DSG2-KD lines demonstrated significantly reduced adhesion to BMEC-DSG2 compared to the NT control cell line (FIG. 6C-D), with shRNA constructs A and C inducing a 28.1% and 35.6% reduction in adhesion, respectively. Based on these findings, we propose that a potential biological role for DSG2 on MM PC is to mediate adhesion to BM endothelium.

Of note, we recently demonstrated that the closely related molecule, N-cadherin, also mediates adhesion of MM PC to BM endothelial cells (34, 35). Similar to DSG2, N-cadherin was found to be expressed on MM PC from a distinct subset of patients. Accordingly, we next examined whether these two cadherins, with apparently similar function, were co-expressed on MM PC. There was a moderate (r=0.26) but statistically significant (p<0.0001) positive correlation between DSG2 and CDH2 gene expression within the entire patient cohort (FIG. 6E). Notably, however, almost all instances of co-expression of these two genes occurred within the MS subgroup (highlighted in red in FIG. 6E), with 57/68 (83.8%) of samples from patients in the MS subgroup expressing both DSG2 and CDH2. In contrast, DSG2 expression was independent of CDH2 in the majority of non-MS patients, with 37/346 (10.7%) expressing DSG2 alone, 97/346 (28.0%) expressing CDH2 alone and just 22/346 (6.4%) co-expressing both DSG2 and CDH2. Thus, DSG2 and CDH2 may be induced together by the NSD2 methyltransferase in patients with the t(4;14) translocation, but are likely subject to independent regulation in non-MS subtype MM PC.

Discussion

In the present study, we identified that DSG2 is a surface protein aberrantly expressed by MM PC in a distinct subset of patients with particularly poor prognosis. The strong association between DSG2 expression and poor prognosis suggests a functional role for DSG2 in MM pathogenesis. To this end, our functional studies demonstrate that DSG2 mediates adhesive interactions between MM PC and BM endothelial cells. We hypothesize that these interactions may contribute to the dissemination of MM PC, by promoting the extravasation of circulating MM PC from the blood into new sites in the BM.

DSG2 is principally involved in the formation of desmosomal adhesion structures. Desmosomes are important for maintaining the integrity of tissues which are subjected to high degrees of mechanical stress, including epithelial tissues and the myocardium. It is therefore counter-intuitive that DSG2 would be expressed by MM PC, which have not been described to form desmosomes, and do not form a tightly integrated tissue structure requiring the strong adhesive forces that desmosomes provide. However, DSG2 is emerging as unique amongst the desmosomal cadherins, with many functions now described in addition to desmosome formation. In the context of cancer. Using the KMS-11 MM cell line, we found that DSG2 knockdown had no measurable effect on proliferation, survival or activation of the NFκB, ERK or AKT signaling pathways. In addition, in the present study we also tested the effect of DSG2 knockdown on the migration/invasion of KMS-11 cells into Matrigel and found no difference between control and knockdown cells.

In the absence of a clear role for DSG2 in the proliferation, survival, signaling or migration of MM PC, we considered the possibility that it may be functioning as an adhesion molecule, independent of desmosome formation. We therefore hypothesized that DSG2 may mediate adhesion to the vascular endothelium via homotypic DSG2-DSG2 interactions, since endothelial cells can also express DSG2 in certain tissues (14, 15). This possibility was further strengthened by our observation that blood vessels within patient BM biopsies expressed DSG2 on their inner lumen, and that the endothelial cell line TrHBMEC, derived from normal human BM, expresses DSG2. Strikingly, reducing expression of DSG2 resulted in a significant decrease in adhesion of KMS-11 MM cells to a monolayer of DSG2+ TrHBMEC cells. While adhesion was not completely blocked, this was unsurprising as several other adhesion molecules have been reported to mediate the adhesion of MM PC to endothelial cells, including integrin α4β1 (38), CD44 (39, 40) and N-cadherin (34). On the basis of these functional studies, we propose that a key function for DSG2 on MM PC is to mediate adhesion to DSG2-expressing endothelial cells.

One of the defining features of MM is the presence of multiple lesions at sites throughout the skeleton at the time of diagnosis (19), suggesting that MM PC dissemination is an intrinsic feature of this cancer. Notably, elevated numbers of circulating tumor cells are a predictor of disease progression from MGUS and smoldering MM (41-43) and disease relapse following therapy (44-46), independent of tumor burden, suggesting the importance of hematogenous spread in MM disease progression. The process of dissemination of MM PC is thought to be similar to that of metastasis in solid tumors, requiring adhesion to vascular endothelial cells to enable transendothelial migration and facilitate spread to secondary sites via the peripheral circulation (47, 48). To this end, several adhesion molecules, including integrin α₄β₁, CD44 and N-cadherin, have been shown to play a role in adhesion of primary MM PC and MM cell lines to endothelial cells in vitro (34, 38-40). Moreover, studies using shRNA targeting or functional inhibitors have demonstrated that blockade of N-cadherin or CD44 is sufficient to inhibit the homing of MM PC from the peripheral blood to the BM in mouse models of MM (34, 39, 49), highlighting the importance of MM PC adhesion to endothelial cells in the dissemination process. Our present observations suggest that circulating MM PC may also use DSG2 to bind to vascular endothelium and thus exit the bloodstream to seed new sites. This mechanism would be expected to contribute to disease progression, and may, at least in part, explain the link between DSG2 expression and poor prognosis.

The results presented here reveal that DSG2 is a clinically useful prognostic biomarker in MM. Being a surface protein detectable by flow cytometry, DSG2 could be readily assessed as part of routine diagnostic analysis of BM specimens to provide valuable prognostic information at the time of diagnosis. The ability to recognize high-risk MM at diagnosis is becoming increasingly important as personalized treatment approaches gain momentum, seen, for example, with the use of upfront tandem autologous stem cell transplantation for genetic high-risk MM resulting in improved clinical outcomes (58). Furthermore, so-called response-adapted approaches are being examined in clinical trials, where therapy is altered based on objective measures such as BM minimal residual disease (MRD) during treatment (59).

Together, our studies suggest that DSG2 is a molecule of great relevance in MM biology. DSG2 plays a non-redundant role in the adhesion of MM PC to endothelial cells, and is thus a potential therapeutic target for reducing or preventing disease dissemination and progression. In addition, the clear link between DSG2 expression and poor prognosis implicates this surface protein as a readily measurable and clinically useful prognostic biomarker, which could be used for guiding treatment decisions and thus optimizing current and emerging therapies.

### Example 2—DSG2 KD Sensitises KMS-11 Cells to a Bortezomib

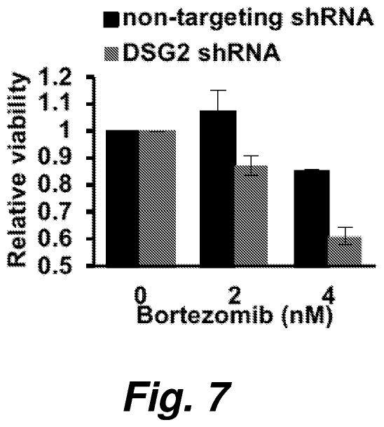

It has also been found that DSG2 KD sensitises KMS-11 cells to frontline therapy with bortezomib. The results are shown in FIG. 7.

Lentiviral vectors (pGIPZ) expressing DSG2-shRNA and non-silencing control-shRNA were obtained from Open Biosystem (Dharmacon) and cloned into the pGIPZ expression plasmid: 5′-TGGATGTCAATGACAATAT-3′ (SEQ ID NO: 2; DSG2-shRNA60); 5′-CCAGTGTTCTACCTAAATA-3′ (SEQ ID NO:3; DSG2-shRNA62); and 5′-ATCTCGCTTGGGCGAGAGTAAG-3′ (SEQ ID NO: 4; non-silencing shRNA). Replication incompetent lentiviral particles were generated by transiently co-transfecting HEK293T cells with ViraPower Lentiviral Support Kit (Invitrogen) and pGIPZ-shRNA vectors using Lipofectamine 2000 (Invitrogen). Lentiviral supernatant was harvested 72 hours post-transfection and used to transduce 1×10⁵ KMS-11 cells that were seeded in a 6-well plate, in the presence of 4 μg/mL polybrene. Puromycin (1 μg/mL) was continually added to maintain culture of cells with the transduced vectors and DSG2 expression was routinely checked using flow cytometry and western blot. Cell without and with DSG2 KD were cultured with increasing concentrations of Bortezomib (2-4 nM, (Janssen Cilag, New Brunswick NJ)) and after 12 hours the cell viability was determined using AnnexinV staining using flow cytometric analysis.

### Example 3—Flow Cytometry can be Used to Replace Genetic Testing

As described above DSG2 is expressed by CD138+ PCs in the bone marrow. In addition, DSG2 can be detected on the surface of CD38+ PC in the bone marrow and peripheral blood.

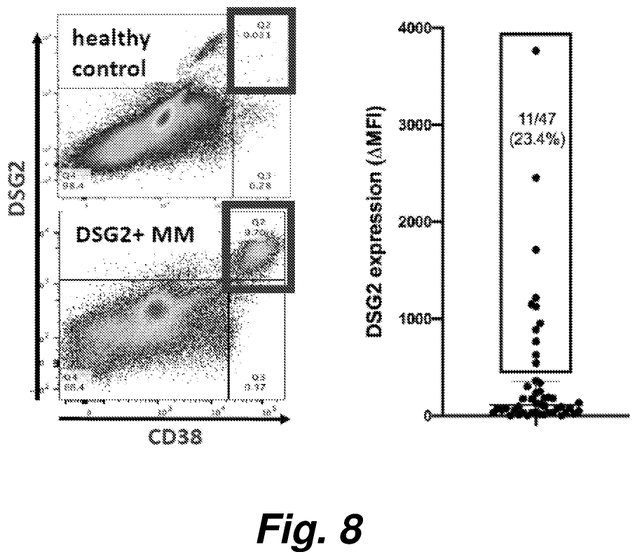

FIG. 8 demonstrates the detection of DSG2+ PCs in bone marrow by FACS for CD38 and DSG2 expression. In healthy controls, few cells are located in quadrant 2. In multiple myeloma patients it can be seen that there is a large number of cells in quadrant 2 cells expressing high levels of DSG2 and CD38.

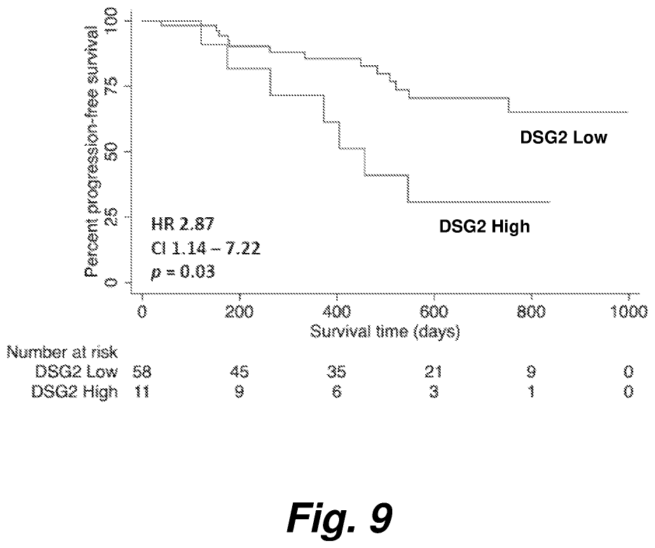

FIG. 9 shows Kaplan-Meier survival estimates using flow cytometry patient data. The data directly shows that the flow cytometry test performs as expected, namely that it correctly predicts prognosis.

### Example 4—Use of DSG2 to Inform Treatment of Patients with Multiple Myeloma

For newly-diagnosed myeloma subjects or those with relapsed/refractory myeloma, the level of DSG2 in the bone marrow, and specifically on the bone marrow plasma cells, will be determined by flow cytometry. Once a bone marrow sample is obtained, this determination can be made within 24 hours and appropriate treatment commenced shortly thereafter, thereby minimising the time to receiving lifesaving therapy. Those subjects found to express high levels of DSG2 on their bone marrow plasma cells will be considered for therapies known to be important for treating high genetic risk and/or poor prognosis myeloma. Such therapies should include proteasome inhibitors (e.g. bortezomib, carfilzomib or ixazomib) and/or monoclonal antibodies (e.g. daratumumab, isatuximab or elotuzumab) with a corticosteroid (e.g. dexamethasone or prednisolone). Immunomodulatory drugs (thalidomide, lenalidomide or pomalidomide) may be added to proteasome inhibitors and/or monoclonal antibodies and corticosteroids for increased efficacy. Methods are known in the art for treating patients with the aforementioned agents.

Subjects found to express low levels of DSG2 on their bone marrow plasma cells will be considered for therapies known to be suitable for treating standard to intermediate genetic risk and/or standard to intermediate prognosis myeloma. Such therapies should include immunomodulatory drugs (thalidomide, lenalidomide or pomalidomide) and a corticosteroid (e.g. dexamethasone or prednisolone) to which proteasome inhibitors (e.g. bortezomib, carfilzomib or ixazomib) and/or monoclonal antibodies (e.g. daratumumab, isatuximab or elotuzumab) could be added to increase efficacy. Methods are known in the art for treating patients with the aforementioned agents.

In addition to the level of DSG2, suitable treatment regimes may also be selected on the basis of clinical and/or biological characteristics of the subject and the subject's myeloma. Clinical characteristics include but are not limited to the subject's age, co-morbidities and performance status, as measured, for example, using the Eastern Cooperative Oncology Group (ECOG) scale. Biological characteristics include but are not limited to the presence and extent/severity of the myeloma defining “CRAB” criteria (hypercalcaemia, renal impairment, anaemia and bone disease), genetic risk stratification of the myeloma, and the presence or otherwise of cytopenias, neuropathy, cardiac disease, amyloidosis and plasma cell leukaemia.

### REFERENCES

1. Kumar S K, Dispenzieri A, Lacy M Q, Gertz M A, Buadi F K, Pandey S, et al. Continued improvement in survival in multiple myeloma: changes in early mortality and outcomes in older patients. Leukemia. 2014; 28:1122-8.
2. Rollig C, Knop S, Bornhauser M. Multiple myeloma. Lancet. 2015; 385:2197-208.
3. Cho H, Yoon D H, Lee J B, Kim S Y, Moon J H, Do Y R, et al. Comprehensive evaluation of the revised international staging system in multiple myeloma patients treated with novel agents as a primary therapy. Am J Hematol. 2017; 92:1280-6.
4. Rajan A M, Rajkumar S V. Interpretation of cytogenetic results in multiple myeloma for clinical practice. Blood Cancer J. 2015;5:e365.
5. Kalff A, Spencer A. The t(4;14) translocation and FGFR3 overexpression in multiple myeloma: prognostic implications and current clinical strategies. Blood Cancer J. 2012;2:e89.
6. Chandesris M O, Soulier J, Labaume S, Crinquette A, Repellini L, Chemin K, et al. Detection and follow-up of fibroblast growth factor receptor 3 expression on bone marrow and circulating plasma cells by flow cytometry in patients with t(4;14) multiple myeloma. Br J Haematol. 2007; 136:609-14.
7. Gonsalves W I, Rajkumar S V, Gupta V, Morice W G, Timm M M, Singh P P, et al. Quantification of clonal circulating plasma cells in newly diagnosed multiple myeloma: implications for redefining high-risk myeloma. Leukemia. 2014; 28:2060-5.
8. Granell M, Calvo X, Garcia-Guinon A, Escoda L, Abella E, Martinez C M, et al. Prognostic impact of circulating plasma cells in patients with multiple myeloma: implications for plasma cell leukemia definition. Haematologica. 2017; 102:1099-104.
9. Schafer S, Stumpp S, Franke W W. Immunological identification and characterization of the desmosomal cadherin Dsg2 in coupled and uncoupled epithelial cells and in human tissues. Differentiation. 1996; 60:99-108.
10. Pieperhoff S, Barth M, Rickelt S, Franke W W. Desmosomal molecules in and out of adhering junctions: normal and diseased States of epidermal, cardiac and mesenchymally derived cells. Dermatol Res Pract. 2010; 2010:139167.
11. Nava P, Laukoetter M G, Hopkins A M, Laur O, Gerner-Smidt K, Green K J, et al. Desmoglein-2: a novel regulator of apoptosis in the intestinal epithelium. Mol Biol Cell. 2007; 18:4565-78.
12. Brennan D, Hu Y, Joubeh S, Choi Y W, Whitaker-Menezes D, O'Brien T, et al. Suprabasal Dsg2 expression in transgenic mouse skin confers a hyperproliferative and apoptosis-resistant phenotype to keratinocytes. J Cell Sci. 2007; 120:758-71.
13. Schmitt C J, Franke W W, Goerdt S, Falkowska-Hansen B, Rickelt S, Peitsch W K. Homo- and heterotypic cell contacts in malignant melanoma cells and desmoglein 2 as a novel solitary surface glycoprotein. J Invest Dermatol. 2007; 127:2191-206.
14. Ebert L M, Tan L Y, Johan M Z, Min K K, Cockshell M P, Parham K A, et al. A non-canonical role for desmoglein-2 in endothelial cells: implications for neoangiogenesis. Angiogenesis. 2016; 19:463-86.
15. Giusti B, Margheri F, Rossi L, Lapini I, Magi A, Serrati S, et al. Desmoglein-2-integrin Beta-8 interaction regulates actin assembly in endothelial cells: deregulation in systemic sclerosis. PLoS One. 2013;8:e68117.
16. Tan L Y, Mintoff C, Johan M Z, Ebert B W, Fedele C, Zhang Y F, et al. Desmoglein 2 promotes vasculogenic mimicry in melanoma and is associated with poor clinical outcome. Oncotarget. 2016.
17. Wang H, Beyer I, Persson J, Song H, Li Z, Richter M, et al. A new human DSG2-transgenic mouse model for studying the tropism and pathology of human adenoviruses. J Virol. 2012; 86:6286-302.
18. Rafii S, Shapiro F, Rimarachin J, Nachman R L, Ferris B, Weksler B, et al. Isolation and characterization of human bone marrow microvascular endothelial cells: hematopoietic progenitor cell adhesion. Blood. 1994; 84:10-9.
19. Rajkumar S V, Dimopoulos M A, Palumbo A, Blade J, Merlini G, Mateos M V, et al. International Myeloma Working Group updated criteria for the diagnosis of multiple myeloma. Lancet Oncol. 2014;15:e538-48.
20. Vandyke K, Zeissig M N, Hewett D R, Martin S K, Mrozik K M, Cheong C M, et al. HIF-2alpha Promotes Dissemination of Plasma Cells in Multiple Myeloma by Regulating CXCL12/CXCR4 and CCR1. Cancer Res. 2017; 77:5452-63.
21. Scott R W, Crighton D, Olson M F. Modeling and imaging 3-dimensional collective cell invasion. J Vis Exp. 2011.
22. Agnelli L, Mosca L, Fabris S, Lionetti M, Andronache A, Kwee I, et al. A SNP microarray and FISH-based procedure to detect allelic imbalances in multiple myeloma: an integrated genomics approach reveals a wide gene dosage effect. Genes Chromosomes Cancer. 2009; 48:603-14.
23. Reme T, Hose D, Theillet C, Klein B. Modeling risk stratification in human cancer. Bioinformatics. 2013; 29:1149-57.
24. Cheong C M, Chow A W, Fitter S, Hewett D R, Martin S K, Williams S A, et al. Tetraspanin 7 (TSPAN7) expression is upregulated in multiple myeloma patients and inhibits myeloma tumour development in vivo. Exp Cell Res. 2015; 332:24-38.
25. Yellapantula V D A, K.; Jelinek, D. F.; Bergsagel, L.; Keats, J. J. The comprehensive genomic characterization of all commercially and non-commercially available multiple myeloma cell lines. Blood. 2013; 122:1914.
26. Zhan F, Huang Y, Colla S, Stewart J P, Hanamura I, Gupta S, et al. The molecular classification of multiple myeloma. Blood. 2006; 108:2020-8.
27. Davis S, Meltzer P S. GEOquery: a bridge between the Gene Expression Omnibus (GEO) and BioConductor. Bioinformatics. 2007; 23:1846-7.
28. Ritchie M E, Phipson B, Wu D, Hu Y, Law C W, Shi W, et al. limma powers differential expression analyses for RNA-sequencing and microarray studies. Nucleic Acids Res. 2015;43:e47.
29. Popovic R, Martinez-Garcia E, Giannopoulou E G, Zhang Q, Zhang Q, Ezponda T, et al. Histone methyltransferase MMSET/NSD2 alters EZH2 binding and reprograms the myeloma epigenome through global and focal changes in H3K36 and H3K27 methylation. PLoS Genet. 2014;10:e1004566.
30. Brito J L, Walker B, Jenner M, Dickens N J, Brown N J, Ross F M, et al. MMSET deregulation affects cell cycle progression and adhesion regulons in t(4;14) myeloma plasma cells. Haematologica. 2009; 94:78-86.
31. Hennigan R F, Hawker K L, Ozanne B W. Fos-transformation activates genes associated with invasion. Oncogene. 1994; 9:3591-600.
32. Kamekura R, Kolegraff K N, Nava P, Hilgarth R S, Feng M, Parkos C A, et al. Loss of the desmosomal cadherin desmoglein-2 suppresses colon cancer cell proliferation through EGFR signaling. Oncogene. 2013.
33. Peitsch W K, Doerflinger Y, Fischer-Colbrie R, Huck V, Bauer A T, Utikal J, et al. Desmoglein 2 depletion leads to increased migration and upregulation of the chemoattractant secretoneurin in melanoma cells. PLoS One. 2014;9:e89491.
34. Mrozik K M, Cheong C M, Hewett D, Chow A W, Blaschuk O W, Zannettino A C, et al. Therapeutic targeting of N-cadherin is an effective treatment for multiple myeloma. Br J Haematol. 2015; 171:387-99.
35. Vandyke K, Chow A W, Williams S A, To L B, Zannettino A C. Circulating N-cadherin levels are a negative prognostic indicator in patients with multiple myeloma. Br J Haematol. 2013; 161:499-507.
36. Cai F, Zhu Q, Miao Y, Shen S, Su X, Shi Y. Desmoglein-2 is overexpressed in non-small cell lung cancer tissues and its knockdown suppresses NSCLC growth by regulation of p27 and CDK2. J Cancer Res Clin Oncol. 2017; 143:59-69.
37. Overmiller A M, McGuinn K P, Roberts B J, Cooper F, Brennan-Crispi D M, Deguchi T, et al. c-Src/Cav1-dependent activation of the EGFR by Dsg2. Oncotarget. 2016; 7:37536-55.
38. Parmo-Cabanas M, Bartolome R A, Wright N, Hidalgo A, Drager A M, Teixido J. Integrin alpha4beta1 involvement in stromal cell-derived factor-1alpha-promoted myeloma cell transendothelial migration and adhesion: role of cAMP and the actin cytoskeleton in adhesion. Exp Cell Res. 2004; 294:571-80.
39. Asosingh K, Gunthert U, De Raeve H, Van Riet I, Van Camp B, Vanderkerken K. A unique pathway in the homing of murine multiple myeloma cells: CD44v10 mediates binding to bone marrow endothelium. Cancer Res. 2001; 61:2862-5.
40. Okada T, Hawley R G, Kodaka M, Okuno H. Significance of VLA-4-VCAM-1 interaction and CD44 for transendothelial invasion in a bone marrow metastatic myeloma model. Clin Exp Metastasis. 1999; 17:623-9.
41. Kumar S, Rajkumar S V, Kyle R A, Lacy M Q, Dispenzieri A, Fonseca R, et al. Prognostic value of circulating plasma cells in monoclonal gammopathy of undetermined significance. J Clin Oncol. 2005; 23:5668-74.
42. Bianchi G, Kyle R A, Larson D R, Witzig T E, Kumar S, Dispenzieri A, et al. High levels of peripheral blood circulating plasma cells as a specific risk factor for progression of smoldering multiple myeloma. Leukemia. 2013; 27:680-5.
43. Gonsalves W I, Rajkumar S V, Dispenzieri A, Dingli D, Timm M M, Morice W G, et al. Quantification of circulating clonal plasma cells via multiparametric flow cytometry identifies patients with smoldering multiple myeloma at high risk of progression. Leukemia. 2017; 31:130-5.
44. Dingli D, Nowakowski G S, Dispenzieri A, Lacy M Q, Hayman S R, Rajkumar S V, et al. Flow cytometric detection of circulating myeloma cells before transplantation in patients with multiple myeloma: a simple risk stratification system. Blood. 2006; 107:3384-8.
45. Chakraborty R, Muchtar E, Kumar S K, Jevremovic D, Buadi F K, Dingli D, et al. Risk stratification in myeloma by detection of circulating plasma cells prior to autologous stem cell transplantation in the novel agent era. Blood Cancer J. 2016;6:e512.
46. Nowakowski G S, Witzig T E, Dingli D, Tracz M J, Gertz M A, Lacy M Q, et al. Circulating plasma cells detected by flow cytometry as a predictor of survival in 302 patients with newly diagnosed multiple myeloma. Blood. 2005; 106:2276-9.
47. Ghobrial I M. Myeloma as a model for the process of metastasis: implications for therapy. Blood. 2012; 120:20-30.
48. Vanderkerken K, De Greef C, Asosingh K, Arteta B, De Veerman M, Vande Broek I, et al. Selective initial in vivo homing pattern of 5T2 multiple myeloma cells in the C57BL/KalwRij mouse. Br J Cancer. 2000; 82:953-9.
49. Groen R W, de Rooij M F, Kocemba K A, Reijmers R M, de Haan-Kramer A, Overdijk M B, et al. N-cadherin-mediated interaction with multiple myeloma cells inhibits osteoblast differentiation. Haematologica. 2011; 96:1653-61.
50. Lauring J, Abukhdeir A M, Konishi H, Garay J P, Gustin J P, Wang Q, et al. The multiple myeloma associated MMSET gene contributes to cellular adhesion, clonogenic growth, and tumorigenicity. Blood. 2008; 111:856-64.
51. Shafraz O, Rubsam M, Stahley S N, Caldara A L, Kowalczyk A P, Niessen C M, et al. E-cadherin binds to desmoglein to facilitate desmosome assembly. Elife. 2018;7.
52. Park J, Son Y, Lee N G, Lee K, Lee D G, Song J, et al. DSG2 Is a Functional Cell Surface Marker for Identification and Isolation of Human Pluripotent Stem Cells. Stem Cell Reports. 2018; 11:115-27.
53. Harkness L, Christiansen H, Nehlin J, Barington T, Andersen J S, Kassem M. Identification of a membrane proteomic signature for human embryonic stem cells independent of culture conditions. Stem Cell Res. 2008; 1:219-27.
54. Eshkind L, Tian Q, Schmidt A, Franke W W, Windoffer R, Leube R E. Loss of desmoglein 2 suggests essential functions for early embryonic development and proliferation of embryonal stem cells. Eur J Cell Biol. 2002; 81:592-8.
55. Zhang S, Cui W. Sox2, a key factor in the regulation of pluripotency and neural differentiation. World J Stem Cells. 2014; 6:305-11.
56. Mallampati S, Sun B, Lu Y, Ma H, Gong Y, Wang D, et al. Integrated genetic approaches identify the molecular mechanisms of Sox4 in early B-cell development: intricate roles for RAG1/2 and CKlepsilon. Blood. 2014; 123:4064-76.
57. Geutskens S B, Andrews W D, van Stalborch A M, Brussen K, Holtrop-de Haan S E, Parnavelas J G, et al. Control of human hematopoietic stem/progenitor cell migration by the extracellular matrix protein Slit3. Lab Invest. 2012; 92:1129-39.
58. Cavo M, Petrucci M T, Di Raimondo F, Zamagni E, Gamberi B, Crippa C, et al. Upfront Single Versus Double Autologous Stem Cell Transplantation for Newly Diagnosed Multiple Myeloma: An Intergroup, Multicenter, Phase III Study of the European Myeloma Network (EMNO2/HO95 MM Trial). Blood. 2016; 128:991-.
59. Baz R, Naqvi SMH, Lee J H, Brayer J, Hillgruber N, Fridley B L, et al. Lenalidomide-based response-adapted therapy for older adults without high risk myeloma. Br J Haematol. 2019; 184:735-43.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A method of prognosis for a subject suffering from multiple myeloma, the method comprising determining the level of desmoglein 2 (DSG2) in malignant plasma cells from the subject, wherein an increased level of DSG2 in the plasma cells is indicative of a poorer prognosis for the subject.

2. The method according to claim 1, wherein the malignant plasma cells are obtained from bone marrow and/or blood.

3. The method according to claim 1, wherein the determining of the level of DSG2 in the plasma cells comprises protein detection, immunological detection, and/or RNA detection.

4. The method according to any one of claims 1 to 3, wherein the determining of the level of DSG2 comprises detecting cell surface expression of DSG2.

5. The method according to claim 4, wherein the detecting of cell surface expression of DSG2 comprises flow cytometry.

6. The method according to any one of claims 1 to 5, wherein the level of DSG2 is based on a difference in mean or median fluorescence intensity between plasma cells as determined by flow cytometry.

7. The method according to claim 6, wherein the difference in fluorescence intensity is determined by comparison to fluorescence intensity of unstained cells and/or reference cells and/or cells stained with an isotype control.

8. The method according to any one of claims 1 to 4, wherein the determining of the level of DSG2 comprises detecting the DSG2 by immunohistochemistry.

9. The method according to any one of claims 1 to 8, wherein the malignant plasma cells comprises plasma cells not having a t(4;14) translocation.

10. The method according to any one of claims 1 to 9, wherein the method comprises producing a prognostic score for the subject.

11. The method according to any one of claims 1 to 10, wherein the method comprises stratifying the prognostic score and assessing the clinical outcome for the subject based on the stratification.

12. The method according to any one of claims 1 to 11, wherein the method comprises using a computer processor means to assess the prognosis.

13. The method according to any one of claims 1 to 12, wherein the method is used for determining response to treatment, determining clinical outcome, determine rate of disease progression, assessing rate of disease progression, determining survival, and selecting a treatment regime for a subject.

14. A method of assessing the response of a subject suffering from multiple myeloma to treatment, the method comprising determining the level of desmoglein 2 (DSG2) in malignant plasma cells from the subject, wherein an increased level of DSG2 in the plasma cells is indicative of a reduced response of the subject to the treatment.

15. A method of assessing clinical outcome for a subject suffering from multiple myeloma, the method comprising determining the level of desmoglein 2 (DSG2) in malignant plasma cells from the subject, wherein an increased level of DSG2 in the plasma cells is indicative of a poor clinical outcome for the subject.

16. A method of assessing progression of multiple myeloma in a subject, the method comprising determining the level of desmoglein 2 (DSG2) in malignant plasma cells from the subject, wherein an increased level of DSG2 in the plasma cells is indicative of progression of the multiple myeloma to a more severe stage in the subject.

17. A method of assessing survival of a subject suffering from multiple myeloma, the method comprising determining the level of desmoglein 2 (DSG2) in malignant plasma cells from the subject, wherein an increased level of DSG2 in the plasma cells is indicative of a reduced period of survival for the subject.

18. A method of selecting a treatment regime for a subject suffering from multiple myeloma, the method comprising determining the level of desmoglein 2 (DSG2) in malignant plasma cells from the subject, wherein the level of DSG2 in the plasma cells is used to select a treatment regime for the subject.

19. Use of desmoglein 2 (DSG2) as a marker for (i) prognosis for a subject suffering from multiple myeloma, (ii) assessing response of a subject suffering from multiple myeloma to treatment, (iii) assessing clinical outcome for a subject suffering from multiple myeloma, (iv) assessing progression of multiple myeloma in a subject, (v) assessing survival of a subject suffering from multiple myeloma, and/or (vi) selecting a treatment regime for a subject suffering from multiple myeloma.

20. Use of an antibody to desmoglein 2 (DSG2) and/or an antigen binding part thereof for (i) prognosis for a subject suffering from multiple myeloma, (ii) assessing response of a subject suffering from multiple myeloma to treatment, (iii) assessing clinical outcome for a subject suffering from multiple myeloma, (iv) assessing progression of multiple myeloma in a subject, (v) assessing survival of a subject suffering from multiple myeloma, and/or (vi) selecting a treatment regime for a subject suffering from multiple myeloma.

21. A kit for performing the method according to any of claims 1 to 17.

22. The kit according to claim 21, wherein the kit comprises an antibody to DSG2 and/or an antigen binding part thereof.

23. The kit according to claim 21 or 22, wherein the kit comprises one or more of an antibody, and/or antigen binding party thereof, for detecting CD138, an antibody, and/or antigen binding party thereof, for detecting CD38, an antibody, and/or antigen binding party thereof, for detecting CD19, and an antibody, and/or antigen binding party thereof, for detecting CD20.

24. A method of prognosis for a subject suffering from multiple myeloma to treatment, the method comprising using an antibody to desmoglein 2 (DSG2) and/or an antigen binding part thereof to assess the level of DSG2 in a bone marrow sample and/or a blood sample from the subject and thereby provide a prognosis for the subject on the basis of the level of DSG2 determined.

25. A method of assessing the response of a subject suffering from multiple myeloma to treatment, the method comprising using an antibody to desmoglein 2 (DSG2) and/or an antigen binding part thereof to assess the level of DSG2 in a bone marrow sample and/or a blood sample from the subject and thereby assessing the response of the subject to treatment on the basis of the level of DSG2 determined.

26. A method of assessing clinical outcome for a subject suffering from multiple myeloma, the method comprising using an antibody to desmoglein 2 (DSG2) and/or an antigen binding part thereof to assess the level of DSG2 in a bone marrow sample and/or a blood sample from the subject and thereby assessing the clinical outcome for the subject on the basis of the level of DSG2 determined.

27. A method of assessing progression of multiple myeloma in a subject, the method comprising using an antibody to desmoglein 2 (DSG2) and/or an antigen binding part thereof to assess the level of DSG2 in a bone marrow sample and/or a blood sample from the subject and thereby assessing the progression of the multiple myeloma in the subject on the basis of the level of DSG2 determined.

28. A method of assessing survival of a subject suffering from multiple myeloma to treatment, the method comprising using an antibody to desmoglein 2 (DSG2) and/or an antigen binding part thereof to assess the level of DSG2 in a bone marrow sample and/or a blood sample from the subject and thereby assessing the survival of the subject on the basis of the level of DSG2 determined.

29. A method of selecting a treatment regime for a subject suffering from multiple myeloma, the method comprising using an antibody to desmoglein 2 (DSG2) and/or an antigen binding part thereof to assess the level of DSG2 in a bone marrow sample and/or a blood sample from the subject and thereby selecting the treatment regime on the basis of the level of DSG2 determined.

30. A computer-readable medium encoded with programming instructions executable by a computer processor means to allow the computer processor means to process data associated with the level of desmoglein 2 (DSG2) in malignant plasma cells and provide a prognosis for a subject suffering from multiple myeloma.

31. A computer processor means comprising a computer-readable medium according to claim 29.

32. A system for providing a prognosis for a subject suffering from multiple myeloma, the system comprising a computer processor having a computer-readable medium encoded with programming instructions executable by the computer processor means to allow the computer processor means to process data associated with the level of desmoglein 2 (DSG2) in malignant plasma cells from the subject and provide a prognosis for the subject.

33. The system according to claim 31, wherein the data is transferred over the internet to the computer processing means.

34. A method of treating a subject suffering from multiple myeloma, the method comprising:
identifying a subject likely to be responsive to a treatment for multiple myeloma on the basis of the level of desmoglein 2 (DSG2) in malignant plasma; and
treating the subject so identified.

35. A method of selecting a specific treatment for a subject suffering from multiple myeloma, the method comprising:
determining the level of desmoglein 2 (DSG2) in malignant plasma cells from the subject; and
identifying a specific treatment for the subject on the basis of the DSG2 determined.

36. A method of treating a subject suffering from multiple myeloma, the method comprising:
determining the level of desmoglein 2 (DSG2) in malignant plasma cells from the subject;
identifying a specific treatment for the subject on the basis of the DSG2 determined; and
treating the subject with the specific treatment.

37. A method of identifying an agent for treating multiple myeloma, the method comprising:
determining the ability of a candidate agent to reduce desmoglein 2 (DSG2) dependent adhesion of malignant plasma cells to endothelial cells; and
identifying the agent as an agent for treating multiple myeloma.
